SEQUENCE LISTING (10 sequences)

SEQ ID NO: 1 - protein, 117 aa, Artificial Sequence [sequence omitted]

SEQ ID NO: 2 - protein, 107 aa, Artificial Sequence [sequence omitted]

SEQ ID NO: 3 - protein, 8 aa, Artificial Sequence
  HCDR1
GFTFSNFG

SEQ ID NO: 4 - protein, 8 aa, Artificial Sequence
  HCDR2
ISGGGRDT

SEQ ID NO: 5 - protein, 10 aa, Artificial Sequence
  HCDR3
VKWGNIYFDY

SEQ ID NO: 6 - protein, 6 aa, Artificial Sequence
  LCDR1
LSINTF

SEQ ID NO: 7 - protein, 3 aa, Artificial Sequence
  LCDR2
AAS

SEQ ID NO: 8 - protein, 9 aa, Artificial Sequence
  LCDR3
QQSSNTPFT

SEQ ID NO: 9 - protein, 444 aa, Artificial Sequence
  HC
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial Sequence
  LC
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0326846 A1
(43) Pub. Date: Oct. 23, 2025

(54) METHODS OF TREATING CANCER IN IMMUNOSUPPRESSED OR IMMUNOCOMPROMISED PATIENTS BY ADMINISTERING A PD-1 INHIBITOR

(71) Applicants: Regeneron Pharmaceuticals, Inc., Tarrytown, NY (US); Sanofi Biotechnology, Paris (FR)

(72) Inventors: Jigar Desai, Pine Brook, NJ (US); Matthew G. Fury, New York, NY (US); Alexander Seluzhytsky, Bridgewater, NJ (US); Nikita Piyush Mehta, Bridgewater, NJ (US)

(21) Appl. No.: 18/551,679

(22) PCT Filed: Mar. 22, 2022
(86) PCT No.: PCT/US2022/071248

§ 371 Date: Sep. 21, 2023

Related U.S. Application Data
(60) Provisional application No. 63/164,662, filed on Mar. 23, 2021.
(60) Provisional application No. 63/183,690, filed on May 4, 2021.
(60) Provisional application No. 63/267,548, filed on Feb. 4, 2022.
(60) Provisional application No. 63/267,797, filed on Feb. 10, 2022.

(30) Foreign Application Priority Data
Jul. 16, 2021 (EP) 21315128.5

(51) Int. Cl. C07K 16/28; A61K 9/00; A61K 39/00; A61K 45/06; A61P 35/00

(52) U.S. Cl. CPC C07K 16/2818; A61K 9/0019; A61K 45/06; A61P 35/00; A61K 2039/505; A61K 2039/54; A61K 2039/545; C07K 2317/565

(57) ABSTRACT

The present disclosure provides methods for treating or inhibiting the growth of a tumor, including selecting a patient with cancer, wherein the patient is immunosuppressed or immunocompromised, and administering to the patient a therapeutically effective amount of a programmed death 1 (PD-1) inhibitor (e.g., an anti-PD-1 antibody, such as cemiplimab or a bioequivalent thereof). In certain embodiments, the cancer is skin cancer, such as cutaneous squamous cell carcinoma.

### CROSS-REFERENCE TO RELATED APPLICATIONS

This application is a U.S. national phase application under 35 U.S.C. § 371 of International Patent Application No. PCT/US2022/071248 filed Mar. 22, 2022, which claims the benefit of priority to U.S. Provisional Patent Application No. 63/267,797 filed Feb. 10, 2022, U.S. Provisional Patent Application No. 63/267,548 filed Feb. 4, 2022, European Patent Convention Patent Application No. 21315128.5 filed Jul. 16, 2021, U.S. Provisional Patent Application No. 63/183,690 filed May 4, 2021, and U.S. Provisional Patent Application No. 63/164,662 filed Mar. 23, 2021, the disclosures of all of which are hereby incorporated by reference in their entireties.

### REFERENCE TO A SEQUENCE LISTING

The sequence listing of the present application is submitted electronically as a computer readable sequence listing in ASCII format with a file name of “SeqList10932-ST25.txt,” a creation date of Feb. 27, 2024, and a size of 8,996 bytes. The sequence listing submitted is part of the specification and is hereby incorporated by reference in its entirety.

### FIELD

The present disclosure relates to methods of treating or inhibiting the growth of a tumor, including selecting an immunosuppressed or immunocompromised patient with cancer in need thereof and administering to the patient a therapeutically effective amount of a programmed death 1 (PD-1) inhibitor (e.g., an anti-PD-1 antibody, such as cemiplimab or a bioequivalent thereof).

### BACKGROUND

Programmed death 1 (PD-1) plays an important role in autoimmunity, tumor immunity and infectious immunity, and is thus an ideal target for immunotherapy. Blocking PD-1 with antagonists, including monoclonal antibodies, has been studied in treatments of cancer and chronic viral infections. Blockade of PD-1 is also an effective and well tolerated approach to stimulating the immune response, and has achieved therapeutic advantage against various human cancers, including melanoma, renal cell cancer (RCC), and non-small cell lung cancer (NSCLC). (Sheridan 2012, Nat. Biotechnol., 30:729-730; Postow et al., 2015, J Clin Oncol, 33:1974-1982; Chen et al., 2013, Nat. Rev. Immunol., 13:227-242; Riley, 2009, Immunol. Rev., 229:114-125; Dong et al., 1999, Nature Med., 5(12): 1365-1369; Zou, 2008, Nat. Rev. Immunol., 8:467-77; Ribas 2012, NEJM 366:2517-2519; Watanabe et al., 2012, Clin. Dev. Immunol. Vol. 2012, Article ID: 269756; Wang et al., 2013, J. Viral Hep., 20:27-39; Flies et al., 2011, Yale J. Biol. Med., 84:409-421; Pardoll, 2012, Nature, 12:252-264; Freeman, 2008, PNAS, 105:10275-10276; Francisco et al., 2010, Immunol. Rev., 236:219-242).

Monoclonal antibodies to PD-1 are known in the art and have been described, for example, in U.S. Pat. Nos. 9,987,500, 8,008,449, 8,168,757, US 20110008369, US 20130017199, US 20130022595, WO 2006121168, WO 20091154335, WO 2012145493, WO 2013014668, WO 2009101611, EP 2262837, and EP 2504028. Cemiplimab (also known as REGN2810; LIBTAYO®), for example, is a high-affinity, fully human, hinge-stabilized IgG4P antibody directed to the PD-1 receptor that potently blocks the interaction of PD-1 with its ligands, PD-L1 and PD-L2.

Skin cancer is the most common cancer in the United States (Guy et al., Am. J. Prev. Med. 48:183-87, 2015). An estimated 5.4 million cases of non-melanoma skin cancer, including basal cell carcinoma and squamous cell carcinoma, were diagnosed in the United States in 2012 (Rogers et al., JAMA Dermatol., 151(10):1081-86, 2015). Cutaneous squamous cell carcinoma (CSCC) is the second-most common malignancy in the United States, after basal cell carcinoma (BCC) (Karia et al., J. Am. Acad. Dermatol. 68:957-66, 2013). Risk factors for CSCC include UV exposure, advanced age, and immunosuppression (Alam et al., New Engl. J. Med. 344:975-83, 2001; Madan, Lancet 375:673-85, 2010). Although the vast majority of individuals diagnosed with CSCC or BCC have a very favorable prognosis, CSCC has a greater propensity for aggressive recurrences than BCC. (Rees et al., Int. J. Cancer 137:878-84, 2015).

Surgical resection is the centerpiece of clinical management of CSCC or BCC. However, some patients who develop advanced CSCC, which encompasses both locally advanced and metastatic CSCC, are not candidates for surgery. Some such patients may be administered post-operative radiation therapy or chemotherapy, but these may be undesirable options due to safety and tolerability concerns. And while the surgical cure rate for CSCC is >95%, some patients have high risk of recurrence as assessed by immune status, primary disease stage, extent of nodal involvement, presence of extracapsular extension, and prior treatment. Postoperative radiation therapy (RT) is recommended for these patients, but relapse with locoregional recurrence or distant metastases may still occur.

Recurrent CSCC increases the risk of subsequent recurrences. In a single institution retrospective study of 212 patients, recurrent CSCCs were twice as likely to recur again after excisional surgery as compared to primary CSCCs (Harris et al., Otolaryngol Head Neck Surg, 156(5):863-69, 2017). (Brantsch et al., Lancet Oncol 9(8):713-20, 2008; Harris et al., Otolaryngol Head Neck Surg, 156(5):863-69, 2017; Thompson et al., JAMA Dermatol 2016; 152(4):419-28, 2016). For patients with unresectable advanced CSCC, the malignancy is a life-threatening condition, even though some patients may achieve durable disease control with radiation-based therapy. (Nottage et al., Journal of Clinical Oncology 2012; 30(15_suppl):8538; Samstein et al., J Skin Cancer 2014; 2014:284582). Regarding systemic therapies, there have been single-arm studies that often contained heterogeneous groups of CSCC patients with different stages of disease, but none of these studies clearly demonstrated therapeutic advantage (Maubec et al., J Clin Oncol, 2011; 29(25):3419-26; Nakamura et al., Int J Clin Oncol, 2013; 18(3):506-9).

The most common clinical subtype of BCC is nodular BCC (Wu et al., 2013, Am J Epidemiol, 178:890-7). Most BCC patients are cured by surgery, but a small percentage of patients experience recurrent lesions or develop unresectable locally advanced or metastatic disease. Recognition of the oncogenic role of the G-protein receptor Smoothened (SMO) in BCC led to the development of vismodegib and sonidegib, orally available inhibitors of SMO, generally referred to as Hedgehog Inhibitors (HHIs). In addition to adverse side-effects of the HHIs, it was found that for patients that progress on one HHI (vismodegib), subsequent treatment with another HHI (sonedegib) did not result in tumor inhibition (Danial et al., Clin. Cancer Res. 22:1325-29, 2016).

Further, some CSCC patients are considered to have high risk CSCC, as assessed using a number of factors, including cancer staging using the American Joint Committee on Cancer, 8th Edition (AJCC, 2017), immune status, lymphovascular invasion, extent of nodal involvement, presence of extracapsular extension and treatment history. Post-operative radiotherapy is recommended in high risk cases (Bichakjian et al., J Natl Compr Canc Netw, 16(6):742-74, 2018) (Stratigos, Eur J Cancer, 51(14):1989-2007, 2015). However, high risk patients may relapse with locoregional recurrence or distant metastases (Porceddu et al., J Clin Oncol, 36(13):1275-83, 2018).

Immunosuppressed and/or immunocompromised patients are at increased risk for solid tumors and cutaneous malignancies with estimated risk of nonmelanoma skin cancer increased by 10-250-fold (Athar et al., Arch Biochem Biophys. 2011;508:159-163). Limited data exist on the safety and effectiveness of immune checkpoint inhibitors (ICIs) in these patients because they are frequently excluded from clinical trials of ICIs. Additionally, transplant recipients are known to be at higher risk for CSCC than for any other tumor type (Euvard et al., New Engl. J. Med., 348(17):1681-91, 2003). CSCC also has a more aggressive clinical course in transplant patients as compared to immunocompetent CSCC patients (Manyam et al., Cancer, 123(11):2054-60, 2017). Systemic administration of PD-1 inhibitors in transplant patients presents a high risk of allograft rejection or injury (Lipson et al., New Engl. J. Med., 374(9):896-98, 2016; Aguirre et al., The Oncologist, 24:394-401, Nov. 9, 2018; Starke et al., Kidney Int., 78(1):38-47, 2010).

Therefore, there remains a need to provide safe and effective therapies for treating cancer in immunosuppressed or immunocompromised patients.

### SUMMARY

In one aspect, the disclosed technology relates to a method of treating or inhibiting the growth of a tumor, including: (a) selecting a patient with cancer, wherein the patient is immunosuppressed or immunocompromised; and (b) administering to the patient a therapeutically effective amount of a programmed death-1 (PD-1) inhibitor. In some embodiments, the cancer is selected from anal cancer, bladder cancer, bone cancer, breast cancer, brain cancer, cervical cancer, colon cancer, colorectal cancer, endometrial cancer, esophageal cancer, head and neck cancer, kidney cancer, liver cancer, lung cancer, myeloma, ovarian cancer, pancreatic cancer, prostate cancer, salivary gland cancer, skin cancer, stomach cancer, testicular cancer, and uterine cancer. In some embodiments, the cancer is skin cancer. In some embodiments, the skin cancer selected from cutaneous squamous cell carcinoma (CSCC), basal cell carcinoma (BCC), Merkel cell carcinoma, and melanoma. In some embodiments, the skin cancer is CSCC. In some embodiments, the skin cancer is metastatic or locally advanced CSCC and the patient is not a candidate for curative surgery or curative radiation.

In some embodiments, the skin cancer is BCC. In some embodiments, the skin cancer is metastatic or locally advanced BCC, and wherein the patient has been previously treated with a hedgehog pathway inhibitor (HHI) or for whom HHI is not appropriate. In some embodiments, the patient is immunocompromised or immunosuppressed due to a history of solid organ transplant. In some embodiments, the patient is immunocompromised or immunosuppressed due to an autoimmune disease or disorder. In some embodiments, the patient is immunocompromised or immunosuppressed due to a hematologic malignancy. In some embodiments, the hematologic malignancy comprises a heme cancer. In some embodiments, the heme cancer is chronic lymphocytic leukemia. In some embodiments, the cancer is CSCC and patient has at least one high-risk feature selected from: (1) nodal disease with (a) extracapsular extension and at least one node ≥20 mm or (b) at least three positive lymph nodes; (2) in-transit metastases; (3) T4 lesion; (4) perineural invasion; and (5) recurrent CSCC with at least one other risk factor. In some embodiments, the PD-1 inhibitor is an antibody or antigen-binding fragment thereof that binds specifically to PD-1, PD-L1 or PD-L2, or a bioequivalent thereof.

In some embodiments, the PD-1 inhibitor is an antibody or antigen-binding fragment thereof that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof. In some embodiments, the anti-PD-1 antibody or antigen-binding fragment thereof includes HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8. In some embodiments, the anti-PD-1 antibody or antigen-binding fragment thereof includes a HCVR including an amino acid sequence of SEQ ID NO: 1. In some embodiments, the anti-PD-1 antibody or antigen-binding fragment thereof includes a LCVR including an amino acid sequence of SEQ ID NO: 2. In some embodiments, the anti-PD-1 antibody or antigen-binding fragment thereof includes a HCVR/LCVR amino acid sequence pair of SEQ ID NOs: 1/2. In some embodiments, the anti-PD-1 antibody includes a heavy chain and a light chain, wherein the heavy chain has an amino acid sequence of SEQ ID NO: 9. In some embodiments, the anti-PD-1 antibody includes a heavy chain and a light chain, wherein the light chain has an amino acid sequence of SEQ ID NO: 10. In some embodiments, the anti-PD-1 antibody includes a heavy chain and a light chain, wherein the heavy chain has an amino acid sequence of SEQ ID NO: 9 and the light chain has an amino acid sequence of SEQ ID NO: 10. In some embodiments, the PD-1 inhibitor is cemiplimab or a bioequivalent thereof.

In some embodiments, the PD-1 inhibitor is an anti-PD-1 antibody or antigen-binding fragment thereof including a HCVR with 90% sequence identity to SEQ ID NO: 1. In some embodiments, the PD-1 inhibitor is an anti-PD-1 antibody or antigen-binding fragment thereof including a LCVR with 90% sequence identity to SEQ ID NO: 2. In some embodiments, the PD-1 inhibitor is an anti-PD-1 antibody or antigen-binding fragment thereof including a HCVR with 90% sequence identity to SEQ ID NO: 1, and a LCVR with 90% sequence identity to SEQ ID NO: 2. In some embodiments, the PD-1 inhibitor is an anti-PD-1 antibody selected from cemiplimab, nivolumab, pembrolizumab, pidilizumab, MEDI0608, BI 754048, PF-06371548, spartalizumab, camrelizumab, JNJ-63313240, and MCLA-134. In some embodiments, the PD-1 inhibitor is an anti-PD-L1 antibody selected from REGN3504, avelumab, atezolizumab, durvalumab, MDX-1105, LY3300054, FAZ053, STI-1014, CX-031, KN035, and CK-301.

In some embodiments, the administration of the PD-1 inhibitor promotes tumor regression, reduces tumor cell load, reduces tumor burden, and/or prevents tumor recurrence in the patient. In some embodiments, the administration of the PD-1 inhibitor leads to at least one effect selected from an increase in one or more of overall response rate, progression-free survival, overall survival, complete response, partial response, and stable disease. In some embodiments, the administration of the PD-1 inhibitor does not cause an adverse event related to the immunosuppressed or immunocompromised condition of the patient. In some embodiments, the PD-1 inhibitor is administered as a monotherapy.

In some embodiments, the PD-1 inhibitor is administered in combination with an additional therapeutic agent or therapy selected from surgery, radiation, an anti-viral therapy, photodynamic therapy, HHI therapy, imiquimod, a programmed death ligand-1 (PD-L1) inhibitor, a lymphocyte activation gene 3 (LAG3) inhibitor, a cytotoxic T-lymphocyte-associated protein 4 (CTLA-4) inhibitor, a glucocorticoid-induced tumor necrosis factor receptor (GITR) agonist, a T-cell immunoglobulin and mucin domain containing protein-3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an antagonist of another T-cell co-inhibitor or ligand, a CD20 inhibitor, an indoleamine-2,3-dioxygenase (IDO) inhibitor, a CD28 activator, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an agonist to a co-stimulatory receptor, an antibody to a tumor-specific antigen, a vaccine, an adjuvant to increase antigen presentation, an oncolytic virus, a cytotoxin, a chemotherapeutic agent, platinum-based chemotherapy, a tyrosine kinase inhibitor, an IL-6R inhibitor, an IL-4R inhibitor, an IL-10 inhibitor, a cytokine, an antibody drug conjugate (ADC), chimeric antigen receptor T cells, an anti-inflammatory drug, a non-steroidal anti-inflammatory drug (NSAID), and a dietary supplement.

In some embodiments, the PD-1 inhibitor is administered as one or more doses, wherein each dose is administered every two weeks, three weeks, four weeks, five weeks or six weeks. In some embodiments, the PD-1 inhibitor is administered as two or more doses, wherein each dose is administered every three weeks. In some embodiments, the PD-1 inhibitor is administered at a dose of 5 mg to 800 mg. In some embodiments, the PD-1 inhibitor is administered at a dose of 200 mg, 250 mg, 350 mg, or 700 mg. In some embodiments, the PD-1 inhibitor is administered at a dose of 1 mg/kg to 20 mg/kg of the patient's body weight. In some embodiments, the PD-1 inhibitor is administered at a dose of 1 mg/kg, 3 mg/kg or 10 mg/kg of the patient's body weight. In some embodiments, the PD-1 inhibitor is administered intravenously, or subcutaneously.

In another aspect, the disclosed technology relates to a programmed death 1 (PD-1) inhibitor for use in a method of treating or inhibiting the growth of a tumor, the method including: (a) selecting a patient with cancer, wherein the patient is immunosuppressed or immunocompromised; and (b) administering to the patient a therapeutically effective amount of a programmed death-1 (PD-1) inhibitor.

In another aspect, the disclosed technology relates to a kit including a programmed death 1 (PD-1) inhibitor in combination with written instructions for use of a therapeutically effective amount of the PD-1 inhibitor for treating or inhibiting the growth of a tumor in an immunosuppressed or immunocompromised cancer patient.

Other embodiments of the present disclosure will become apparent from the detailed description below.

### BRIEF DESCRIPTION OF THE DRAWINGS

FIG. 1 is a schematic of the CemiplimAb-rwlc Survivorship and Epidemiology study described in Example 2. Abbreviations: NRS, numeric rating scale; PRO, patient-reported outcomes; QLQ-C30, quality of life questionnaire-core30; QLQ-ELD14, quality of life questionnaire for elderly patients; SCI, skin care index; SEBI, sun exposure behavior inventory.

FIG. 2 is a bar graph showing duration of exposure of patients included in the study described in Example 2.

FIG. 3 is a schematic of the design for Part 1 of the study described in Example 3.

### DETAILED DESCRIPTION

It is to be understood that the present disclosure is not limited to the particular methods and experimental conditions described, as such methods and conditions may vary. It is also to be understood that the terminology used herein is for the purpose of describing particular embodiments only, and is not intended to be limiting, and that the scope of the present disclosure will be limited only by the appended claims. Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are hereby incorporated by reference in their entirety unless otherwise stated.

### Methods of Treating or Inhibiting Growth of Cancer

In general, immunosuppressed or immunocompromised cancer patients, such as those who have received an organ transplant, constitute an underrepresented subpopulation that is often excluded from clinical trials. Transplant recipients, for example require particularly close monitoring to avoid potential rejection of the transplant during administration of the therapy being studied. However, the present disclosure includes effective methods for treating or inhibiting the growth of a tumor in an immunosuppressed or immunocompromised patient with cancer by administering to the patient in need thereof a PD-1 inhibitor, such as cemiplimab or a bioequivalent thereof. Surprisingly, the disclosed methods achieve anti-tumor efficacy in immunosuppressed or immunocompromised cancer patients even when the PD-1 inhibitor is administered systemically. It is further surprising that such efficacy is achieved without diminishing the safety or quality of life of the patient—e.g., without causing an increased incidence of adverse events arising from the patient's immunosuppressed or immunocompromised condition. For instance, in some embodiments, the disclosed methods may be used to effectively treat or inhibit the growth of a tumor in an immunosuppressed or immunocompromised cancer patient who has received an organ transplant without causing transplant rejection or adverse events related thereto. Increasing the patient's safety profile and quality of life by avoiding adverse events related to the patient's immunosuppressed or immunocompromised condition is a particularly advantageous aspect of the disclosed methods and satisfies a long felt and previously unmet need in this vulnerable patient population.

As used herein, “immunosuppressed” or “immunocompromised” refers to having a weakened immune system, wherein the patient has a reduced ability to fight disease and infection. An immunocompromised condition may be caused by a variety of circumstances, such as certain diseases or ailments (e.g., cancer including heme cancers, AIDS, diabetes, viral infections), malnutrition, stress, and genetic disorders. An immunocompromised condition may also be created, for example, by immunosuppression that is intended to prevent a patient's immune system from responding to an antigen. Non-limiting examples of immunosuppressed or immunocompromised patients include transplant recipients, patients diagnosed with and/or undergoing therapy for an autoimmune disease, patients with a hematologic malignancy (e.g., heme cancer, such as leukemia, including chronic lymphocytic leukemia (CLL)), and patients undergoing chemotherapy. In general, transplant recipients are immunosuppressed in order to prevent the rejection of transplanted cells (e.g., bone marrow, skin cells, endothelial cells, etc.), tissue, or organ (e.g., solid organ) received by the patient from a donor. In accordance with the present disclosure, a patient that is immunosuppressed or immunocompromised may be immunosuppressed, immunocompromised or both.

As used herein, the terms “treating”, “treat”, or the like, mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for surgery, and/or to increase duration of survival of the subject. In many embodiments, the terms “tumor”, “lesion,” “tumor lesion,” “cancer,” and “malignancy” are used interchangeably and refer to one or more cancerous growths.

As used herein, the term “recurrent” refers to a frequent or repeated diagnosis of cancer in a patient or a frequent or repeated occurrence of individual tumors, such as primary tumors and/or new tumors that may represent recurrence of a prior tumor. In certain embodiments, administration of the PD-1 inhibitor inhibits the recurrence of a cancer tumor in the patient.

As used herein, the expression “a subject in need thereof” means a human or non-human mammal that is immunosuppressed or immunocompromised and exhibits one or more symptoms or indications of cancer and/or who has been diagnosed with cancer, and who needs treatment for the same. In many embodiments, the terms “subject” and “patient” are used interchangeably. The expression includes patients who are transplant recipients, such as those who have received transplanted cells (e.g., bone marrow, skin cells, endothelial cells, etc.), tissue, or organ (e.g., solid organ) from a donor, or patients with a history of solid organ transplant. The expression also includes patients with an autoimmune disorder, hematologic malignancy (e.g., heme cancer, such as leukemia, including CLL), or other condition or disease that leads to the subject having a weakened immune system. The expression also includes patients with primary, established, metastatic, or recurrent tumors (advanced malignancies)—e.g., a human patient diagnosed with a primary or a metastatic tumor and/or with one or more symptoms or indications including, but not limited to, unexplained weight loss, general weakness, persistent fatigue, loss of appetite, fever, night sweats, bone pain, shortness of breath, swollen abdomen, chest pain/pressure, enlargement of spleen, and elevation in the level of a cancer-related biomarker (e.g., CA125). The expression also includes subjects with primary or established tumors. The expression also includes immunocompromised human subjects that have and/or need treatment for a solid tumor, e.g., anal cancer, bladder cancer, bone cancer, breast cancer, brain cancer, cervical cancer, colon cancer, colorectal cancer, endometrial cancer, esophageal cancer, head and neck cancer, kidney cancer, liver cancer, lung cancer, myeloma, ovarian cancer, pancreatic cancer, prostate cancer, salivary gland cancer, skin cancer (e.g., BCC, CSCC, Merkel cell carcinoma, and melanoma), stomach cancer, testicular cancer, and uterine cancer.

In certain embodiments, the expression “a subject in need thereof” includes immunosuppressed or immunocompromised patients with a liquid or solid tumor that is resistant to or refractory to or is inadequately controlled by prior therapy (e.g., treatment with an anti-cancer agent). For example, the expression includes subjects who have been treated with one or more lines of prior therapy such as treatment with chemotherapy (e.g., carboplatin or docetaxel), surgery, and/or radiation. The expression also includes patients with a liquid or solid tumor that has been treated with one or more lines of prior therapy but which has subsequently relapsed or metastasized. For example, patients with a liquid or solid tumor that may have received treatment with one or more anti-cancer agents leading to tumor regression; however, subsequently have relapsed with cancer resistant to the one or more anti-cancer agents (e.g., chemotherapy-resistant cancer, HHI-resistant cancer) are treated with the methods of the present disclosure. The expression also includes subjects with a liquid or solid tumor for which conventional anti-cancer therapy is inadvisable, for example, due to toxic side effects. For example, the expression includes patients who have received one or more cycles of HHI with toxic side effects. In specific embodiments, the expression includes human subjects who have and/or need treatment for locally advanced or metastatic cancer. In certain embodiments, the expression includes patients with a liquid or solid tumor that is resistant to, refractory to, or inadequately controlled by prior therapy (e.g., surgery, chemotherapy, radiation, treatment with a different anti-cancer agent (e.g., an anti-cancer agent other than cemiplimab or a bioequivalent thereof) or a combination thereof). In certain embodiments, the expression includes subjects with cancer (e.g., skin cancer) who are not candidates for surgical resection or definitive chemoradiation. In certain embodiments, the expression includes cancer patients with a chronic viral infection caused by a virus, such as human immunodeficiency virus (HIV), hepatitis B virus (HBV), hepatitis C virus (HCV), human papillomavirus (HPV), cytomegalovirus (CMV), or a combination thereof. In certain embodiments, the expression includes patients with one or more of the following diagnoses in their medical history: allogenic bone marrow transplant, solid organ transplant, HIV, inflammatory bowel disease, leukemia, lupus, lymphoma, multiple myeloma, multiple sclerosis, psoriasis or psoriatic arthritis, rheumatoid arthritis, polycythemia vera, myeloproliferative disorder, and chronic obstructive pulmonary disease (COPD) with prednisone.

As used herein, “skin cancer” refers to cancer of the skin, such as basal cell carcinoma (BCC), cutaneous squamous cell carcinoma (CSCC), Merkel cell carcinoma, and melanoma. In some embodiments, the skin cancer is a non-melanoma skin cancer—e.g., BCC, CSCC, or Merkel cell carcinoma. In some embodiments, the skin cancer is cutaneous squamous cell carcinoma (CSCC) or basal cell carcinoma (BCC). In some embodiments, the skin cancer is metastatic CSCC (mCSCC) or locally advanced CSCC (laCSCC)—e.g., unresectable laCSCC. In some embodiments, the skin cancer is laCSCC and the patient is not a candidate for curative surgery or curative radiation. In some embodiments, the skin cancer is metastatic BCC (mBCC) or locally advanced BCC (laBCC). In some embodiments, the skin cancer is laBCC and the patient has been previously treated with a hedgehog pathway inhibitor or for whom a hedgehog pathway inhibitor is not appropriate—e.g., the laBCC has progressed on, or the laBCC patient was intolerant to, hedgehog inhibitor (HHI) therapy.

As used herein, “lung cancer” refers to cancer of the lung, such as non-small cell lung cancer (NSCLC) (e.g., advanced NSCLC, stage IIIB, stage IIIC, or stage IV squamous or non-squamous NSCLC, adenocarcinoma, squamous cell carcinoma, or large cell carcinoma), adenosquamous carcinoma, and sarcomatoid carcinoma. In some embodiments, the lung cancer is non-small cell lung cancer. In some embodiments, the lung cancer is squamous non-small cell lung cancer. In some embodiments, the lung cancer is non-squamous non-small cell lung cancer. In some embodiments, the lung cancer is locally advanced, recurrent or metastatic lung cancer. In some embodiments, the patient has lung cancer wherein the tumors express PD-L1 in ≥50% of tumor cells. In some embodiments, the patient has lung cancer (e.g., non-small cell lung cancer) wherein the tumors express PD-L1 in ≥50%, ≥60%, ≥70%, ≥80%, or ≥90% of tumor cells. In some embodiments, the patient has been previously treated with a treatment for lung cancer (e.g., an anti-tumor therapy such as chemotherapy, radiation, or a combination thereof).

In certain embodiments, the methods of the present disclosure are used for treating a subject with a solid tumor. As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumors. The term includes different types of solid tumors named for the cell types that form them, viz. sarcomas, carcinomas and blastomas. In certain embodiments, the term “solid tumor” refers to cancers including, but not limited to, anal cancer, angiosarcoma, basal cell carcinoma, bladder cancer, bone cancer, brain cancer, breast cancer, cervical cancer, cholangiocarcinoma, chondrosarcoma, colon cancer, colorectal cancer, cutaneous squamous cell carcinoma, endometrial cancer, esophageal cancer, glioblastoma multiforme, head and neck squamous cell cancer, hepatocellular carcinoma, kidney cancer, liver cancer, lung cancer, Merkel cell carcinoma, melanoma, myeloma, non-small cell lung cancer, ovarian cancer, pancreatic cancer, prostate cancer, salivary gland cancer, skin cancer, soft tissue sarcoma, stomach cancer, testicular cancer, and uterine cancer.

In certain embodiments, the methods of the present disclosure are used for treating a subject with a liquid tumor. As used herein, the term “liquid tumor” refers to cancerous cells present in body fluids or soft tissue, such as blood or bone marrow. The expression “liquid tumor” includes cancers arising from connective or supporting tissue (e.g., bone or muscle) (referred to as sarcomas), cancers arising from the body's glandular cells and epithelial cells which line body tissues (referred to as carcinomas), and cancers of the lymphoid organs such as lymph nodes, spleen and thymus (referred to as lymphomas). Lymphoid cells occur in almost all tissues of the body and therefore, lymphomas may develop in a wide variety of organs. In some embodiments, the disclosed methods are used for treating a subject with a liquid tumor comprising a lymphoma or leukemia.

In certain embodiments, the disclosed methods include administering a therapeutically effective amount of a PD-1 inhibitor (e.g., cemiplimab or a bioequivalent thereof) in combination with an additional therapeutic agent or therapy. The additional therapeutic agent or therapy may be administered for increasing anti-tumor efficacy, for reducing toxic effects of one or more therapies and/or for reducing the dosage of one or more therapies. In various embodiments, the additional therapeutic agent or therapy may include one or more of: surgery, radiation, an anti-viral therapy (e.g., cidofovir), photodynamic therapy, HHI therapy (e.g., vismodegib, sonedegib), imiquimod, a programmed death ligand-1 (PD-L1) inhibitor (e.g., an anti-PD-L1 antibody as disclosed in US 2015/0203580 or atezolizumab), a lymphocyte activation gene 3 (LAG3) inhibitor (e.g., an anti-LAG3 antibody), a cytotoxic T-lymphocyte-associated protein 4 (CTLA-4) inhibitor (e.g., ipilimumab), a glucocorticoid-induced tumor necrosis factor receptor (GITR) agonist (e.g., an anti-GITR antibody), a T-cell immunoglobulin and mucin domain containing protein-3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an antagonist of another T-cell co-inhibitor or ligand (e.g., an antibody to CD-28, 2B4, LY108, LAIR1, ICOS, CD160 or VISTA), a CD20 inhibitor (e.g., an anti-CD20 antibody, or a bispecific CD3/CD20 antibody), an indoleamine-2,3-dioxygenase (IDO) inhibitor, a CD28 activator, a vascular endothelial growth factor (VEGF) antagonist (e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, pazopanib, or ramucirumab)), an angiopoietin-2 (Ang2) inhibitor, a transforming growth factor beta (TGFβ) inhibitor, an epidermal growth factor receptor (EGFR) inhibitor (e.g., erlotinib, cetuximab), an agonist to a co-stimulatory receptor (e.g., an agonist to CD28, 4-1BB, or OX40), an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin or a cancer vaccine), an adjuvant to increase antigen presentation (e.g., granulocyte-macrophage colony- stimulating factor), an oncolytic virus, a cytotoxin, a chemotherapeutic agent (e.g., pemetrexed, dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, topotecan, irinotecan, vinorelbine, and vincristine), platinum-based chemotherapy (e.g., platinum-doublet chemotherapy), a tyrosine kinase inhibitor (e.g., lenvatinib, regorafenib, and cabozantinib), an IL-6R inhibitor, an IL-4R inhibitor, an IL-10 inhibitor, a cytokine such as IL-2, IL-7, IL-12, IL-21, and IL-15, an antibody drug conjugate (ADC) (e.g., anti-CD19-DM4 ADC, and anti-DS6-DM4 ADC), chimeric antigen receptor T cells (e.g., CD19-targeted T cells), an anti-inflammatory drug such as a corticosteroid, a non-steroidal anti-inflammatory drug (NSAID), and a dietary supplement such as an antioxidant.

As used herein, the term “anti-viral therapy” refers to any agent, drug or therapy used to treat, prevent, or ameliorate a viral infection in a host subject, including but not limited to: zidovudine, lamivudine, abacavir, ribavirin, lopinavir, efavirenz, cobicistat, tenofovir, rilpivirine, analgesics, corticosteroids, and combinations thereof.

In certain embodiments, administering to an immunocompromised subject with cancer a therapeutically effective amount of a PD-1 inhibitor (e.g., cemiplimab or a bioequivalent thereof) leads to increased inhibition of tumor growth (e.g., tumor regression, tumor shrinkage and/or disappearance) in the treated subject.

In certain embodiments, the administration of a PD-1 inhibitor leads to one or more of: (i) delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years in the treated subject, as compared to an untreated subject or a subject treated with a different anti-cancer therapy or agent (e.g., an anti-cancer therapy other than cemiplimab or a bioequivalent thereof); (ii) increased disease-free survival (DFS) from date of treatment until recurrence of tumor or death, as compared to an untreated subject or a subject treated with a different anti-cancer therapy or agent (e.g., an anti-cancer therapy other than cemiplimab or a bioequivalent thereof); and (iii) improved overall response rate (ORR), complete response (CR), or partial response (PR), as compared to an untreated subject or a subject treated with a different anti-cancer therapy or agent (e.g., an anti-cancer therapy other than cemiplimab or a bioequivalent thereof).

In certain embodiments, administering a therapeutically effective amount of a PD-1 inhibitor (e.g., cemiplimab or a bioequivalent thereof) to an immunocompromised cancer patient prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months as compared to an untreated subject or a subject treated with a different anti-cancer therapy or agent (e.g., an anti-cancer therapy other than cemiplimab or a bioequivalent thereof).

In certain embodiments, administering a therapeutically effective amount of a PD-1 inhibitor (e.g., cemiplimab or a bioequivalent thereof) to an immunocompromised cancer patient leads to increased overall survival (OS) or progression-free survival (PFS) of the subject as compared to a subject treated with a different anti-cancer therapy or agent (e.g., an anti-cancer therapy other than cemiplimab or a bioequivalent thereof). In certain embodiments, the PFS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject treated with chemotherapy alone. In certain embodiments, the OS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject treated with a different anti-cancer therapy or agent (e.g., an anti-cancer therapy other than cemiplimab or a bioequivalent thereof).

### PD-1 Inhibitors

The methods disclosed herein include administering a therapeutically effective amount of a PD-1 inhibitor. As used herein, a “PD-1 inhibitor” refers to any molecule capable of inhibiting, blocking, abrogating or interfering with the activity or expression of PD-1. In some embodiments, the PD-1 inhibitor can be an antibody, a small molecule compound, a nucleic acid, a polypeptide, or a functional fragment or variant thereof. Non-limiting examples of suitable PD-1 inhibitor antibodies include anti-PD-1 antibodies and antigen-binding fragments thereof, anti-PD-L1 antibodies and antigen-binding fragments thereof, and anti-PD-L2 antibodies and antigen-binding fragments thereof. Other non-limiting examples of suitable PD-1 inhibitors include RNAi molecules such as anti-PD-1 RNAi molecules, anti-PD-L1 RNAi, and an anti-PD-L2 RNAi, antisense molecules such as anti-PD-1 antisense RNA, anti-PD-L1 antisense RNA, and anti-PD-L2 antisense RNA, and dominant negative proteins such as a dominant negative PD-1 protein, a dominant negative PD-L1 protein, and a dominant negative PD-L2 protein. Some examples of the foregoing PD-1 inhibitors are described in e.g., U.S. Pat. Nos. 9,308,236, 10,011,656, and US 20170290808.

The term “antibody,” as used herein, is intended to refer to immunoglobulin molecules included of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is included of a heavy chain variable region (“HCVR” or “VH”) and a heavy chain constant region (included of domains CH1, CH2 and CH3). Each light chain is included of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In certain embodiments, the FRs of the antibody (or antigen binding fragment thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules.

As used herein, the terms “antigen-binding fragment” of an antibody, “antigen-binding portion” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically include at least one variable domain. The variable domain may be of any size or amino acid composition and will generally include at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (Xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may include a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The antibodies used in the methods disclosed herein may be human antibodies. As used herein, the term “human antibody” refers to antibodies having variable and constant regions derived from human germline immunoglobulin sequences. The human antibodies of the present disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods disclosed herein may be recombinant human antibodies. As used herein, the term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

In some embodiments, PD-1 inhibitors used in the methods disclosed herein are antibodies or antigen-binding fragments thereof that specifically bind PD-1. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present disclosure, includes antibodies that bind PD-1 or a portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

In some embodiments, the PD-1 inhibitor is a bioequivalent of an anti-PD-1 antibody or antigen-binding fragment thereof. As used herein, the term “bioequivalent” refers to anti-PD-1 antibodies or PD-1-binding proteins or fragments thereof that are pharmaceutical equivalents or pharmaceutical alternatives whose rate and/or extent of absorption do not show a significant difference with that of a reference antibody (e.g., cemiplimab) when administered at the same molar dose under similar experimental conditions, either single dose or multiple dose. In the context of the present disclosure, the term “bioequivalent” includes antigen-binding proteins that bind to PD-1 and do not have clinically meaningful differences with the reference antibody (e.g., cemiplimab) with respect to safety, purity and/or potency.

According to certain embodiments, the PD-1 inhibitor is an anti-PD-1 antibody (e.g., cemiplimab) including three heavy chain complementarity determining regions (HCDRs) of a heavy chain variable region (HCVR) including the amino acid sequence of SEQ ID NO: 1 and three light chain complementarity determining regions (LCDRs) of a light chain variable region (LCVR) including the amino acid sequence of SEQ ID NO: 2. According to certain embodiments, the anti-PD-1 antibody (e.g., cemiplimab) includes three HCDRs (HCDR1, HCDR2 and HCDR3) and three LCDRs (LCDR1, LCDR2 and LCDR3), wherein the HCDR1 includes the amino acid sequence of SEQ ID NO: 3; the HCDR2 includes the amino acid sequence of SEQ ID NO: 4; the HCDR3 includes the amino acid sequence of SEQ ID NO: 5; the LCDR1 includes the amino acid sequence of SEQ ID NO: 6; the LCDR2 includes the amino acid sequence of SEQ ID NO: 7; and the LCDR3 includes the amino acid sequence of SEQ ID NO: 8. In certain embodiments, the anti-PD-1 antibody (e.g., cemiplimab) includes an HCVR including SEQ ID NO: 1 and an LCVR including SEQ ID NO: 2. In certain embodiments, the anti-PD-1 antibody (e.g., cemiplimab) includes a heavy chain including the amino acid sequence of SEQ ID NO: 9 and a light chain including the amino acid sequence of SEQ ID NO: 10. An exemplary anti-PD-1 antibody for use in the disclosed methods is cemiplimab.

Other anti-PD-1 antibodies that can be used in the context of the methods of the present disclosure include, e.g., the antibodies referred to and known in the art as nivolumab, pembrolizumab, MEDI0608, pidilizumab, BI 754091, spartalizumab (also known as PDR001), camrelizumab (also known as SHR-1210), JNJ-63723283, MCLA-134, or any of the anti-PD-1 antibodies set forth in U.S. Pat. Nos. 6,808,710, 7,488,802, 8,008,449, 8,168,757, 8,354,509, 8,609,089, 8,686,119, 8,779,105, 8,900,587, and 9,987,500, and in patent publications WO2006/121168, WO2009/114335. The portions of all of the aforementioned publications that identify anti-PD-1 antibodies are hereby incorporated by reference.

According to certain embodiments, a bioequivalent of cemiplimab is an anti-PD-1 antibody including a HCVR having 90%, 95%, 98% or 99% sequence identity to SEQ ID NO: 1. According to certain embodiments, a bioequivalent of cemiplimab is an anti-PD-1 antibody including a LCVR having 90%, 95%, 98% or 99% sequence identity to SEQ ID NO: 2. According to certain embodiments, a bioequivalent of cemiplimab is an anti-PD-1 antibody including a HCVR having 90%, 95%, 98% or 99% sequence identity to SEQ ID NO: 1, and a LCVR having 90%, 95%, 98% or 99% sequence identity to SEQ ID NO: 2. Sequence identity may be measured by methods known in the art (e.g., GAP, BESTFIT, and BLAST).

According to certain embodiments, a bioequivalent of cemiplimab is an anti-PD-1 antibody including a HCVR including an amino acid sequence of SEQ ID NO: 1 having 1-15 or more amino acid substitutions. According to certain embodiments, a bioequivalent of cemiplimab is an anti-PD-1 antibody including a LCVR including an amino acid sequence of SEQ ID NO: 2 having 1-10 or more amino acid substitutions. According to certain embodiments, a bioequivalent of cemiplimab is an anti-PD-1 antibody including a HCVR including an amino acid sequence of SEQ ID NO: 1 having 1-15 or more amino acid substitutions, and a LCVR including an amino acid sequence of SEQ ID NO: 2 having 1-10 or more amino acid substitutions.

The present disclosure also includes use of anti-PD-1 antibodies or antigen-binding fragments thereof comprising variants of any of the HCVR, LCVR and/or CDR amino acid sequences disclosed herein having one or more conservative amino acid substitutions. For example, the present disclosure includes use of anti-PD-1 antibodies or antigen-binding fragments thereof having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

In some embodiments, PD-1 inhibitors used in the methods disclosed herein are antibodies or antigen-binding fragments thereof that specifically bind PD-L1. For example, an antibody that “specifically binds” PD-L1, as used in the context of the present disclosure, includes antibodies that bind PD-L1 or a portion thereof with a K_{D} of about 1×10⁻⁸ M or less (e.g., a smaller K_{D} denotes a tighter binding). A “high affinity” anti-PD-L1 antibody refers to those mAbs having a binding affinity to PD-L1, expressed as K_{D} of at least 10⁻⁸ M, preferably 10⁻⁹ M, more preferably 10⁻¹⁰ M, even more preferably 10⁻¹¹ M, even more preferably 10⁻¹² M, as measured by surface plasmon resonance, e.g., BIACORE™ or solution-affinity ELISA. An isolated antibody that specifically binds human PD-L1 may, however, have cross-reactivity to other antigens, such as PD-L1 molecules from other (non-human) species.

An exemplary anti-PD-L1 antibody for use in the disclosed methods is REGN3504. Other anti-PD-L1 antibodies that can be used in the disclosed methods include, e.g., the antibodies referred to and known in the art as MDX-1105, atezolizumab (TECENTRIQ™), durvalumab (IMFINZI™), avelumab (BAVENCIO™), LY3300054, FAZ053, STI-1014, CX-072, KN035 (Zhang et al., Cell Discovery, 3, 170004 (March 2017)), CK-301 (Gorelik et al., American Association for Cancer Research Annual Meeting (AACR), 2016 Apr. 4 Abstract 4606), or any of the other anti-PD-L1 antibodies set forth in patent publications U.S. Pat. Nos. 7,943,743, 8,217,149, 9,402,899, 9,624,298, 9,938,345, WO 2007005874, WO 2010077634, WO 2013181452, WO 2013181634, WO 2016149201, WO 2017034916, or EP3177649.

### Pharmaceutical Compositions and Administration

The present disclosure provides therapeutic pharmaceutical compositions including the PD-1 inhibitors disclosed herein. Such pharmaceutical compositions may be formulated with suitable pharmaceutically acceptable carriers, excipients, buffers, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al., “Compendium of excipients for parenteral formulations” PDA, J Pharm Sci Technol 52:238-311 (1998).

The dose of PD-1 inhibitor (e.g., anti-PD-1 antibody) may vary depending upon the age and the size of a subject to be administered, target disease, conditions, route of administration, and the like. When a PD-1 inhibitor of the present disclosure is used for treating or inhibiting the growth of cancer, it may be advantageous to administer the PD-1 inhibitor at a single dose of about 0.1 to about 100 mg/kg body weight. Depending on the severity of the condition, the frequency and the duration of the treatment can be adjusted. In certain embodiments, the PD-1 inhibitor of the present disclosure can be administered as an initial dose of at least about 0.1 mg to about 800 mg, about 1 to about 600 mg, about 5 to about 500 mg, or about 10 to about 400 mg. In certain embodiments, the initial dose may be followed by administration of a second or a plurality of subsequent doses of the PD-1 inhibitor in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. The pharmaceutical composition can be also delivered in a vesicle, in particular a liposome (see, e.g., Langer (1990) Science 249:1527-1533).

The use of nanoparticles to deliver the PD-1 inhibitor of the present disclosure is also contemplated herein. Antibody-conjugated nanoparticles may be used both for therapeutic and diagnostic applications. Antibody-conjugated nanoparticles and methods of preparation and use are described in detail by Arruebo et al., 2009, “Antibody-conjugated nanoparticles for biomedical applications,” J. Nanomat., Vol. 2009, Article ID 439389, 24 pages. Nanoparticles may be developed and conjugated to antibodies contained in pharmaceutical compositions to target cells. Nanoparticles for drug delivery have also been described in, for example, U.S. Pat. Nos. 8,257,740 or 8,246,995.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracranial, and intramuscular injections, drip infusions, etc. These injectable preparations may be prepared by methods publicly known.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. In some embodiments, the amount of the antibody contained is generally about 5 to about 600 mg per dosage form in a unit dose, such as about 5 to about 350 mg, or about 10 to about 300 mg.

In certain embodiments, the present disclosure provides a pharmaceutical composition or formulation including a therapeutic amount of a PD-1 inhibitor (e.g., cemiplimab or a bioequivalent thereof) and a pharmaceutically acceptable carrier. Non-limiting examples of pharmaceutical compositions including an anti-PD-1 antibody provided herein that can be used in the context of the present disclosure are disclosed in US 2019/0040137.

The present disclosure also provides kits comprising a PD-1 inhibitor (e.g., cemiplimab or a bioequivalent thereof) for therapeutic uses as described herein. Kits typically include a label indicating the intended use of the contents of the kit and instructions for use. As used herein, the term “label” includes any writing, or recorded material supplied on, in or with the kit, or which otherwise accompanies the kit. Accordingly, this disclosure provides a kit for treating an immunosuppressed or immunocompromised patient afflicted with a cancer, the kit comprising: (a) a therapeutically effective dosage of a PD-1 inhibitor antibody (e.g., cemiplimab or a bioequivalent thereof); and (b) instructions for using the PD-1 inhibitor in any of the methods disclosed herein.

### Administration Regimens

In certain embodiments, the methods disclosed herein include administering to the tumor of a subject in need thereof a therapeutically effective amount of a PD-1 inhibitor (e.g., cemiplimab or a bioequivalent thereof) in multiple doses, e.g., as part of a specific therapeutic dosing regimen. For example, the therapeutic dosing regimen may include administering one or more doses of a PD-1 inhibitor to the subject at a frequency of about once a day, once every two days, once every three days, once every four days, once every five days, once every six days, once a week, once every two weeks, once every three weeks, once every four weeks, once every five weeks, once every six weeks, once every eight weeks, once every twelve weeks, once a month, once every two months, once every three months, once every four months, twice a day, twice every two days, twice every three days, twice every four days, twice every five days, twice every six days, twice a week, twice every two weeks, twice every three weeks, twice every four weeks, twice every five weeks, twice every six weeks, twice every eight weeks, twice every twelve weeks, twice a month, twice every two months, twice every three months, twice every four months, three times a day, three times every two days, three times every three days, three times every four days, three times every five days, three times every six days, three times a week, three times every two weeks, three times every three weeks, three times every four weeks, three times every five weeks, three times every six weeks, three times every eight weeks, three times every twelve weeks, three times a month, three times every two months, three times every three months, three times every four months or less frequently or as needed so long as a therapeutic response is achieved.

In certain embodiments, the one or more doses are administered in at least one treatment cycle—e.g., 1, 2, 3, 4, 5, 6, 7, 8, 9, or 10 treatment cycles. In certain embodiments, each dose of the PD-1 inhibitor includes 0.1, 1, 0.3, 3, 4, 5, 6, 7, 8, 9 or 10 mg/kg of the patient's body weight. In certain embodiments, each dose includes about 5 to 800 mg of the PD-1 inhibitor, for example about 5, 10, 15, 20, 25, 40, 45, 50, 60, 70, 80, 90, 100, 150, 200, 250, 300, 350, 400, 450, 500, 550, 600, 650, 700, 750 mg or more of the PD-1 inhibitor.

### Dosage

The amount of PD-1 inhibitor (e.g., cemiplimab or a bioequivalent thereof) administered to a subject according to the methods disclosed herein is, generally, a therapeutically effective amount. As used herein, the term “therapeutically effective amount” means an amount of a PD-1 inhibitor administered to an immunocompromised patient for treating cancer that results in one or more of: (a) inhibition of tumor growth, or an increase in tumor necrosis, tumor shrinkage and/or tumor disappearance; (b) a reduction in the severity or duration of a symptom or an indication of cancer—e.g., a tumor lesion; (c) delay in tumor growth and development; (d) inhibition of tumor metastasis; (e) prevention of recurrence of tumor growth; (f) increase in survival of a subject with cancer; and/or (g) delay of surgery, each as compared to an untreated subject or a subject treated with a different anti-cancer therapy or agent (e.g., an anti-cancer therapy other than cemiplimab or a bioequivalent thereof). In certain embodiments, the term refers an amount of a PD-1 inhibitor administered to an immunocompromised patient for treating cancer that results in one or more of the foregoing effects and also maintains the safety or quality of life of the patient with respect to the patient's immunosuppressed or immunocompromised condition. For example, in addition to one or more of the foregoing effects, a “therapeutically effective amount” maintains the safety profile of the patient and does not cause an adverse event or adverse side effect related to the immunosuppressed or immunocompromised patient's organ transplant, autoimmune disease, hematologic malignancy (e.g., heme cancer, such as leukemia, including CLL), chemotherapy or other condition or treatment that has weakened the patient's immune system, even when the PD-1 inhibitor is administered systemically.

In certain embodiments, a therapeutically effective amount of the PD-1 inhibitor (e.g., cemiplimab or a bioequivalent thereof) can be from about 0.05 mg to about 800 mg, from about 1 mg to about 600 mg, from about 10 mg to about 550 mg, from about 50 mg to about 400 mg, from about 75 mg to about 350 mg, or from about 100 mg to about 300 mg of the antibody. For example, in various embodiments, the amount of the PD-1 inhibitor is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg.

The amount of a PD-1 inhibitor (e.g., cemiplimab or a bioequivalent thereof) contained within an individual dose may be expressed in terms of milligrams of antibody per kilogram of subject body weight (i.e., mg/kg). In certain embodiments, the PD-1 inhibitor used in the methods disclosed herein may be administered to a subject at a dose of about 0.0001 to about 100 mg/kg of subject body weight. In certain embodiments, an anti-PD-1 antibody may be administered at dose of about 0.1 mg/kg to about 20 mg/kg of a patient's body weight. In certain embodiments, the methods of the present disclosure include administration of a PD-1 inhibitor (e.g., an anti-PD-1 antibody) at a dose of about 1 mg/kg to 3 mg/kg, 1 mg/kg to 5 mg/kg, 1 mg/kg to 10 mg/kg, 1 mg/kg, 3 mg/kg, 5 mg/kg, or 10 mg/kg of a patient's body weight.

In certain embodiments, an individual dose amount of a PD-1 inhibitor (e.g., cemiplimab or a bioequivalent thereof) administered to a patient may be less than a therapeutically effective amount, i.e., a subtherapeutic dose. For example, if the therapeutically effective amount of a PD-1 inhibitor includes 3 mg/kg, a subtherapeutic dose includes an amount less than 3 mg/kg, e.g., 2 mg/kg, 1.5 mg/kg, 1 mg/kg, 0.5 mg/kg or 0.3 mg/kg. As defined herein, a “subtherapeutic dose” refers to an amount of the PD-1 inhibitor that does not lead to a therapeutic effect by itself. However, in certain embodiments, multiple subtherapeutic doses of a PD-1 inhibitor are administered to collectively achieve a therapeutic effect in the subject.

In certain embodiments, each dose includes 0.1-10 mg/kg (e.g., 0.3 mg/kg, 1 mg/kg, 3 mg/kg, or 10 mg/kg) of PD-1 inhibitor (e.g., cemiplimab or a bioequivalent thereof) based on the subject's body weight. In certain other embodiments, each dose includes 5 to 800 mg of the PD-1 inhibitor, e.g., 5 mg, 10 mg, 15 mg, 20 mg, 25 mg, 30 mg, 40 mg, 45 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, or 800 mg.

### EXAMPLES

The following examples are put forth so as to provide those of ordinary skill in the art with a complete disclosure and description of how to make and use the methods and compositions of the present disclosure and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Study of Cemiplimab for the Treatment of CSCC

This study is a multi-center, non-interventional, longitudinal survivorship cohort study of adult patients with CSCC who receive treatment with commercially available cemiplimab in real-world clinical settings (i.e., outside of an interventional clinical trial). Patients are followed for up to 3 years after being enrolled in the study. This study is designed to collect long-term data regarding the characteristics and survivorship of adult patients with CSCC who receive cemiplimab, and to characterize real-world use patterns, and effectiveness of cemiplimab for CSCC.

Cemiplimab is a high-affinity, human, hinge-stabilized IgG4 monoclonal antibody to the PD-1 receptor that potently blocks the interactions of PD-1 with PD-L1 and PD-L2. Cemiplimab comprises a heavy chain having the amino acid sequence of SEQ ID NO: 9 and a light chain having the amino acid sequence of SEQ ID NO: 10; an HCVR/LCVR amino acid sequence pair comprising SEQ ID NOs: 1/2; and heavy and light chain CDR sequences (HCDR1, HCDR2, HCDR3, LCDR1, LCDR2, LCDR3) comprising SEQ ID NOs: 3-8, respectively, as described herein. See also U.S. Pat. No. 9,987,500.

Prior studies (NCT012383212 and NCT01760498) of cemiplimab in patients with metastatic (nodal or distant) CSCC or laCSCC who were not candidates for curative surgery or curative radiation excluded patients with autoimmune disease that required systemic therapy with immunosuppressant agents within 5 years; history of solid organ transplant; and infection with human immunodeficiency virus (HIV), hepatitis B or hepatitis C. Consequently, data in key populations with advanced CSCC has previously been lacking due to exclusion of these patients from registrational studies (e.g., chronic lymphocytic leukemia [CLL], Immunocompromised); and thus there is a need to expand the available clinical evidence on treatment outcomes for advanced CSCC.

Objectives: Objectives of this study include: (i) to describe the effectiveness of cemiplimab 350 mg administered every 3 weeks (Q3W) for treatment of patients with advanced CSCC in real-world clinical settings; (ii) to evaluate the safety of cemiplimab based on incidence of immune-related adverse events (irAEs), infusion related reactions (IRRs), and treatment related serious adverse reactions (SARs) in patients with advanced CSCC receiving cemiplimab treatment in real world clinical settings; (iii) to describe patient experience, including patient reported quality of life (QOL) and functional status, and clinician reported performance status in a real-world setting for patients with CSCC; (iv) to describe baseline characteristics that could potentially be associated with health-related outcomes for patients with CSCC undergoing treatment with cemiplimab; (v) to describe patients who receive cemiplimab as treatment for CSCC in a real-world setting; (vi) to describe real-world use patterns of cemiplimab for CSCC; (vii) to investigate the long-term effects and effectiveness of cemiplimab in CSCC patients; (viii) to describe the effectiveness of cemiplimab in immunosuppressed and immunocompetent patients with advanced CSCC, regardless of etiology, per available data; (ix) to describe the effectiveness of cemiplimab after prior exposure to radiation therapy for CSCC per available data; and (x) to describe the effectiveness of cemiplimab as first-line (1L) or later systemic treatment in patients with advanced CSCC, regardless of etiology, per available data.

Study Description: The effectiveness patient population will be those who have received cemiplimab and have been assessed for response (stable disease [SD], partial response [PR], complete response [CR], progressive disease [PD]) by a physician. The duration of follow up in the study for each patient will be up to 36 months. At each participating site, all CSCC patients who are currently receiving cemiplimab in a real-world setting, or who will initiate treatment with cemiplimab in a real-world setting, will be screened and offered the opportunity to participate in the study until the enrollment goal is achieved. A minimum of 250 patients with a target of 350 patients will be enrolled at up to 100 study sites. Enrollment will not exceed 500 patients. Each patient will be considered to have completed the study at the time they complete 3 years of follow up or at the time of death.

Study Population: Patients in this study include men and women ≥18 years of age who have recently initiated, or who plan to initiate treatment with commercially available cemiplimab for CSCC in a real-world setting. Participating sites will enroll patients who receive treatment with cemiplimab in real-world settings outside of an interventional clinical trial. Patients who meet all inclusion criteria, none of the exclusion criteria (see below), and sign the informed consent will be included in the study.

Inclusion Criteria: A patient must meet all of the following criteria to be eligible for inclusion in the study: (1) 18 years of age; (2) eligible for treatment with and prescribed cemiplimab for advanced CSCC in accordance with approved prescribing information, (a) patients who are continuing treatment with cemiplimab after completing cemiplimab treatment on the R2810-ONC-1540 clinical trial are eligible to participate in this study at the time that they initiate treatment with cemiplimab in a real-world setting; (b) for completeness and ease of prospective data collection, it is recommended that patients be enrolled prior to administration of their third dose of cemiplimab; (3) willing and able to comply with standard clinical care for advanced CSCC; (4) able to understand and complete study-related questionnaires; (5) provide signed informed consent.

Exclusion Criteria: Patients are not eligible for the study if they meet any of the following criteria: (1) receiving cemiplimab for an indication other than CSCC; (2) any condition that may interfere with patient's ability to participate in the study, (e.g., unstable social situation such as homelessness or psychiatric conditions making follow-up unreliable such as schizophrenia, advanced depression, active substance abuse, or severe cognitive impairment or other comorbidities) that would predictably limit compliance with the intended treatment plan, or prevent the patient from adequately completing QOL assessments; (3) patients concurrently participating in any study including administration of any investigational drug (including cemiplimab) or procedure (including survival follow up).

### Assessments of Patient Experience

Health-related quality of life, including functional status and disease related symptoms, will be captured at baseline and follow-up visits by the following measures: European Organisation for Research and Treatment of Cancer (EORTC) Quality of Life Questionnaire Core Module 30 (EORTC QLQ-C30) and Module for Elderly Cancer Patients (EORTC QLQ-ELD14); Skin Care Index (SCI); Pain as measured by the Pain Numerical Rating Scale (NRS); and Sun Exposure Behaviour Inventory (SEBI). The following information will be collected to determine study eligibility or characterize the baseline population: demographics, medical/surgical history, and completion of the SEBI questionnaire.

The SEBI is a brief self-administered questionnaire that provides useful measures of past and present sun exposure, and current sun behavior, which are used in in studies of skin cancer incidence and risk modification (Jennings et al., J Eur Acad Dermatol Venereol, 2013; 27(6):706-15). Patients will complete this study at baseline only.

The SCI is a 15-item disease-specific QOL instrument, validated for patients with cervicofacial non-melanoma skin cancer (NMSC). It is used to assess behavior modification and risk perceptions in patients with NMSC (Rhee et al., Arch Facial Plast Surg, 2006; 8(5):314-8). Patients will complete this assessment at the time of informed consent, then on day 1 of cycles 3, 5, and 8, then every 3 months during the first 2 years, and every 6 months during the third year. The measures for assessment under SCI include: 3 scales (emotional, social, appearance).

The Pain NRS is a simple assessment tool that patients will complete to report pain at its worst and on average, during the past week. Patients will complete the Pain NRS questionnaire prior to treatment, prior to administration of other tests or procedures, and prior to any discussion of their health status (Williamson et al., J Clin Nurs, 2005; 14(7):798-804). Pain will be repeatedly measured by the Pain NRS at patients' baseline visit and then on day 1 of treatment cycles as outlined below.

The QOL of patients will be repeatedly measured by the EORTC QLC-C30 and EORTC QLQ-ELD14 at their baseline visit and then on day 1 of treatment cycles as outlined below. The measures for assessment under EORTC QLQ-C30 include: global health status, 5 functional scales (physical, role, emotional, cognitive, social), 3 symptom scales, 6 individual symptoms. The measures for assessment under EORTC QLQ-ELD14 include: 5 scales (mobility, worries about others, worries about future, maintaining purpose, burden of illness).

For SCI, Pain NRS, EORTC QLQ-C30, and EORTC QLQ-ELD14, all questionnaires must be completed by the patient prior to treatment administration on cycles 3, 5, 8, 13, 17, 21, 25, 29, 33, 41, and 49. If patients discontinue cemiplimab but remain on study, questionnaires should be completed at SOC follow up visits approximately every 3 months. Each infusion of cemiplimab is considered 1 cycle of treatment.

Other Procedures and Assessments: Other procedures and assessments to be carried out in this study include: physical examination; Eastern Cooperative Oncology Group (ECOG) assessment; targeted history/review of systems (ROS); and cemiplimab IV administration (at least one dose is required). Additional procedures and assessments that may be carried out in this study include: hematology and blood chemistries; radiographic disease assessment (CT using RECIST 1.1 or WHO criteria, PET, or MRI scan, or X-ray); medical photography; clinical disease assessment; concomitant medications; other CSCC interventions post-initiation of cemiplimab; and SARs/irAEs/IRR.

### Adverse Events and Adverse Reactions

An adverse event (AE) is any untoward medical occurrence in a patient administered the study drug which may or may not have a causal relationship with the study drug. Therefore, an AE is any unfavorable and unintended sign (including abnormal laboratory finding), symptom, or disease which is temporally associated with the use of a study drug, whether or not considered related to the study drug (ICH E2A Guideline. Clinical Safety Data Management: Definitions and Standards for Expedited Reporting, October 1994).

An adverse reaction is defined as an AE that is suspected to be related to the medicinal product. That means that a causal relationship between a medicinal product and an AE is at least a reasonable possibility. (ICH E2A Guideline. Clinical Safety Data Management: Definitions and Standards for Expedited Reporting, October 1994).

A serious adverse event (SAE) is any untoward medical occurrence that at any dose: (i) results in death—includes all deaths, even those that appear to be completely unrelated to study drug (e.g., a car accident in which a patient is a passenger); (ii) is life-threatening—the patient is at immediate risk of death at the time of the event. This does not include an AE that had it occurred in a more severe form, might have caused death; (iii) requires in-patient hospitalization or prolongation of existing hospitalization. In-patient hospitalization is defined as admission to a hospital or an emergency room for longer than 24 hours. Prolongation of existing hospitalization is defined as a hospital stay that is longer than was originally anticipated for the event or is prolonged due to the development of a new AE; (iv) results in persistent or significant disability/incapacity (substantial disruption of one's ability to conduct normal life functions); (v) is a congenital anomaly/birth defect; (vi) is an important medical event—Important medical events may not be immediately life-threatening or result in death or hospitalization, but may jeopardize the patient or may require intervention to prevent one of the other serious outcomes listed above (e.g., intensive treatment in an emergency room or at home for allergic bronchospasm; blood dyscrasias or convulsions that do not result in hospitalization; or development of drug dependency or drug abuse). However, pre-planned (prior to signing the ICF) procedures, treatments requiring hospitalization for pre-existing conditions that do not worsen in severity, and admission for palliative or social care are not SAEs; and hospitalization or death due solely to manifestations consistent with typical progression of underlying malignancy is not an SAE.

A serious adverse reaction (SAR) is an adverse drug reaction (ADR) that is treatment related and that met any of the serious criteria of an SAE.

Immune related adverse events (irAEs) are AEs with no other known etiology associated with treatment with anti-PD-1/PD-L1 (including cemiplimab) and other immune checkpoint inhibitors therapies; and consistent with an immune phenomenon. Immune-related AEs which may be severe or fatal, can occur in any organ system or tissue. While irAEs usually manifest during treatment with PD-1/PD-L1 blocking antibodies, irAEs can also manifest after discontinuation of PD-1/PD-L1 blocking antibodies. Examples of irAEs include but are not limited to pneumonitis, colitis, hepatitis, immune skin reactions, immune endocrinopathies (hypothyroidism, hyperthyroidism, adrenal insufficiency, thyroiditis, hypophysitis, type 1 diabetes mellitus), nephritis, encephalitis, meningitis, Guillain Barre syndrome, myasthenia gravis, etc. See approved USPI for further details.

An infusion related reaction (IRR) is defined as any ADR that occurs during cemiplimab infusion or within 24 hours after the infusion is completed. Signs and symptoms usually develop during, or within 24 hours after drug infusion and generally resolve completely within 24 hours of onset. Common symptoms of IRR include fever, chills, cough, tachycardia, hypotension, wheezing, and rash. Other severe forms of IRR may include anaphylaxis and shock. The severity of AEs and IRRs are graded using the current NCI-CTCAE v5.0 grading system or, if not listed in the NCI-CTCAE v5.0, are graded according to Table 1.

TABLE 1
Adverse Event Severity Scale

| | |
|---|---|
| 1 (Mild) | Mild AE (minor; no specific medical intervention; asymptomatic laboratory |
| | findings only, radiographic findings only; marginal clinical relevance) |
| 2 (Moderate) | Moderate AE (minimal intervention; local intervention; noninvasive |
| | intervention [packing, cautery]) |
| 3 (Severe) | Severe and undesirable AE (significant symptoms requiring hospitalization |
| | or invasive intervention; transfusion; elective interventional radiological |
| | procedure; therapeutic endoscopy or operation) |
| 4 (Life- | Life-threatening or disabling AE (complicated by acute, life threatening |
| threatening) | metabolic or cardiovascular complications such as circulatory failure, |
| | hemorrhage, sepsis. Life-threatening physiologic consequences; need for |
| | intensive care or emergent invasive procedure; emergent interventional |
| | radiological procedure, therapeutic endoscopy or operation) |
| 5 (Death) | Death associated with an AE. |

Efficacy outcomes will be assessed in terms of ORR, DCR, DOR, Time to response, PFS, OS, TTTF, and DSD (Table 2). ORR, DCR, DSD, CR, PR, and SD, will be reported in terms of number and percentage of patients along with 95% CI. DOR and TTTF will be summarized by median and range and displayed by Kaplan-Meier approach. PFS and OS will be summarized by median (if observed) and displayed by Kaplan-Meier approach. PFS and OS rates will be reported at milestone time points (3 months, 6 months, 9 months, 12 months, and every 6 months thereafter until 36 months).

TABLE 2
Efficacy Outcomes

| Outcome | Definition |
|---|---|
| Objective response rate (ORR) | the rate of complete responses (CR) or partial responses |
| | (PR) and may include radiographic imaging photography |
| | and/or symptom resolution/clinical assessment. |
| Disease control rate (DCR) | the percentage of patients who have achieved CR, PR or |
| | stable disease (SD) to cemiplimab. |
| Duration of response (DOR) | time from the time of initial response until documented |
| | tumor progression death or initiation of non-cemiplimab |
| | CSCC treatment |
| Time to response | time from date of first administration of cemiplimab to the |
| | initial response. |
| Progression free survival (PFS) | time from date of first administration of cemiplimab to |
| | progression or death from any cause whichever occurs |
| | first. |
| Overall survival (OS) | time from the date of first administration of cemiplimab to |
| | the date of death due to any cause. |
| Time to treatment failure (TTTF) | time from date of first administration of cemiplimab to |
| including lack of response and | treatment discontinuation for disease progression |
| discontinuation due to AE | treatment toxicity or death. |
| Disease specific death (DSD) rate | rate of death caused by or related to underlying CSCC. |

Subgroup Analysis: For ORR, PFS, DOR, and OS outcomes, analyses will be performed for the following subgroups: (i) immunosuppressed patients with advanced CSCC, regardless of etiology; (ii) non-immunosuppressed patients with advanced CSCC, regardless of etiology; (iii) immuno-compromised patients with advanced CSCC, regardless of etiology; (iv) patients treated with cemiplimab as first line (1L) treatment; (v) patients treated with cemiplimab as second line (2L) or later-line treatment; (vi) patients with prior exposure to radiation therapy.

### Example 2: Results of Study of Cemiplimab Treatment in Immunosuppressed and/or Immunocompromised Patients with Advanced CSCC

This example provides results from a CemiplimAb-rwlc Survivorship and Epidemiology (C.A.S.E.) study of immunosuppressed and/or immunocompromised (IS/IC) patients with advanced CSCC. Such patients are at increased risk for solid tumors and cutaneous malignancies. Limited data exist on the safety and effectiveness of immune checkpoint inhibitors (ICIs) in these patients because they are frequently excluded from clinical trials. This example describes the safety and effectiveness results from a cohort of immunosuppressed and/or immunocompromised patients with advanced CSCC enrolled in the C.A.S.E. study (NCT03836105).

The objectives of this study include: (i) describing the effectiveness of cemiplimab 350 mg administered every 3 weeks (Q3W) for treatment of patients with advanced CSCC in real-world clinical settings; (ii) evaluating the safety of cemiplimab based on incidence of treatment-related immune-related adverse events (irAEs), infusion-related reactions (IRRs), and treatment-related serious adverse reactions (TSARs) in patients with advanced CSCC in real-world clinical settings; and (iii) investigating the long-term effectiveness and quality of life (QoL) of cemiplimab in patients with CSCC.

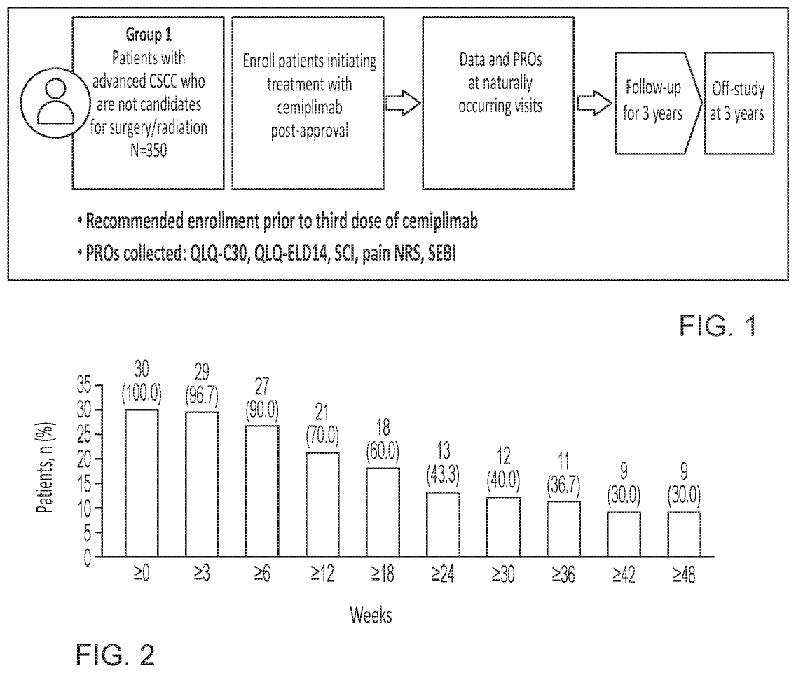

Methods: C.A.S.E. is a prospective, real-world, multi-center, non-interventional, longitudinal study evaluating the effectiveness, safety, quality of life, and survivorship in patients with advanced CSCC treated with cemiplimab. A schematic of the design of this study is provided in FIG. 1. Patients received cemiplimab 350 mg intravenously every 3 weeks per routine standard of care. Patient demographics, disease characteristics, immunosuppression, and relevant medical history were collected. Immunosuppressive regimens varied amongst patients. Investigator assessment of objective response rate (ORR), safety, and tolerability was conducted.

All comers treated for advanced CSCC per clinical judgment were included in the study. IS/IC patients were identified as having one or more of the following diagnoses in medical history: allogenic bone marrow transplant, solid organ transplant, human immunodeficiency virus (HIV), inflammatory bowel disease, leukemia, lupus, lymphoma, multiple myeloma, multiple sclerosis, psoriasis or psoriatic arthritis, rheumatoid arthritis, polycythemia vera, myeloproliferative disorder, and chronic obstructive pulmonary disease (COPD) with prednisone. Clinical activity and safety endpoints include objective response rate (ORR), disease control rate (DCR), treatment-related irAEs, IRRs, and TSARs.

Results: 138 patients were enrolled in the C.A.S.E. study, of which 30 patients were IS/IC based on clinical-reported co-morbidities and/or medication use. For the 30 IS/IC patients, median age was 75.7 years [range: 50-90] and 80% were male (Table 3).

TABLE 3
Baseline demographics, tumor characteristics, and prior treatments

| | IS/IC patients | |
|---|---|---|
| N (%), unless otherwise stated | (N = 30) | |
| Age, years, median (range) | 75.7 | (50-90) |
| Male | 24 | (80.0) |
| ECOG performance status | | |
| 0 | 5 | (16.7) |
| 1 | 18 | (60.0) |
| 2 | 3 | (10.0) |
| Missing | 4 | (13.3) |
| Metastatic CSCC | 11 | (36.7) |
| Locally advanced CSCC | 19 | (63.3) |
| Location of CSCC lesion | | |
| Head and neck | 23 | (76.7) |
| Thorax and abdomen | 3 | (10.0) |
| Upper and lower extremities | 8 | (26.7) |
| Patients with prior surgery | 24 | (80.0) |
| Patients with prior radiation | 17 | (56.7) |
| Patients receiving cemiplimab as 1L | 10 | (33.3) |
| Patients receiving cemiplimab as 2L+ | 20 | (66.7) |
| Multidisciplinary input | 12 | (40.0) |

1L, first-line; 2L, second-line; ECOG, Eastern Cooperative Oncology Group.

The study population included six IS/IC patients who had received a solid organ transplant (n=6, 20%), 14 patients with a hematologic malignancy (n=14, 47%), and 10 patients with an autoimmune disorder (n=10, 33%) (Table 4).

Nine patients had a duration of exposure ≥48 weeks (FIG. 2). Median duration of cemiplimab exposure was 21.6 weeks (interquartile range: 9.9-48.1, range: 0-83).

TABLE 4
Individual IS/IC details of patients

| | | | | Number of |
|---|---|---|---|---|
| | | | Immunosuppression | cemiplimab |
| Patient | Age/ | | (relevant concomitant | infusions in |
| number | sex | Reason for IS/IC | medications) | C.A.S.E. study |
| 1 | 72M | Kidney transplant | Tacrolimus, Sirolimus | 8 |
| 2 | 50M | Kidney transplant | Everolimus, Prednisone | 12 |
| 3 | 78M | Liver transplant | Tacrolimus | 4 |
| 4 | 70M | Liver transplant | Tacrolimus | 22 |
| 5 | 74M | Kidney transplant | Mycophenolate | 7 |
| 6 | 71M | Kidney transplant | Prednisone | 13 |
| 7 | 90M | NHL | Hydroxyurea | 5 |
| 8 | 73M | Small lymphocytic lymphoma | Megestrol, Rituximab | 1 |
| 9 | 86M | B-cell lymphoma | Indolent | 4 |
| 10 | 74M | CLL | Rituximab, Bendamustine | 4 |
| 11 | 85M | CLL | Indolent | 1 |
| 12 | 83M | CLL | Indolent | 4 |
| 13 | 78M | CLL | Indolent | 7 |
| 14 | 67M | CLL | Indolent | 2 |
| 15 | 75M | CLL | Indolent | 11 |
| 16 | 73M | CLL | Indolent | 4 |
| 17 | 79F | Acute promyelocytic leukemia | Indolent | 7 |
| 18 | 82M | Myeloproliferative disorder | Ruxolitinib | 19 |
| 19 | 86M | Polycythemia vera | Fedratinib | 2 |
| 20 | 65M | Polycythemia vera | Fedratinib | 4 |
| 21 | 81F | Psoriasis | Infliximab | 3 |
| 22 | 86F | Psoriasis | Methotrexate | 5 |
| 23 | 73M | Rheumatoid arthritis | Tofacitinib (stopped) | 8 |
| 24 | 73F | Rheumatoid arthritis | Prednisone | 16 |
| 25 | 72M | Rheumatoid arthritis | Methotrexate | 1 |
| 26 | 88F | Rheumatoid arthritis | Methotrexate | 2 |
| 27 | 89F | Polymyalgia rheumatica | Prednisone | 26 |
| 28 | 56M | COPD | Prednisone | 25 |
| 29 | 70M | HIV | N/A | 5 |
| 30 | 71M | HIV | Bictegravir | 15 |
| | | | Emtricitabine-Tenofovir | |

CLL, chronic lymphocytic leukemia; F, female; M, male; N/A, not available; NHL, non-Hodgkin lymphoma.

The median number of treatment cycles was 6.5 (Table 5).

TABLE 5
Number of treatment cycles

| Cycles | IS/IC patients (N = 30) |
|---|---|
| Mean (SD) | 8.2 (6.5) |
| Median | 6.5 |
| Q1:Q3 | 4.0:10.0 |
| Min:Max | 1:25 |

Q, quarter;
SD, standard deviation

As shown in Table 6, ORR was 45.5% (95% confidence interval [CI]: 24.4-67.8. DCR was 63.6% (95% CI: 40.7-82.8).

TABLE 6
Tumor response to cemiplimab

| | IS/IC patients (N = 22)* | |
|---|---|---|
| ORR, % (95% CI) | 45.5 | (24.4-67.8) |
| Best overall response, n (%) | | |
| Complete response | 0 | (0) |
| Partial response | 10 | (45.5) |
| Stable disease | 4 | (18.2) |
| Progressive disease | 2 | (9.1) |
| DCR, % (95% CI) | 63.6 | (40.7-82.8) |

*Eight patients were excluded in the analysis as informed consent and study enrollment were obtained after the third dose of cemiplimab. Six out of the eight patients responded to treatment (two patients responded after enrollment and four patients responded prior to enrollment).

Two out of six patients with indolent CLL had a partial response, an additional two had stable disease, and two patient response assessments were not yet completed. One patient discontinued treatment due to an adverse event and eight patients discontinued treatment due to any reason (including death or withdrawal from the study). Four deaths were reported; none were deemed related or attributable to cemiplimab (one death was due to sepsis, one due to hypoxia, one due to pneumonia, and one due to an unknown cause).

In total, six patients (20%) experienced a treatment-related irAE of any grade regardless of attribution. irAEs experienced by patients include fatigue, pruritis of the forehead and chest, increased alanine aminotransferase, increased aspartate aminotransferase, increased blood creatinine, decreased lymphocyte count, hypothyroidism, and acute renal failure. One TSAR was reported. There were no IRRs. No treatment-related deaths were reported.

Conclusions: The safety, tolerability, and effectiveness of cemiplimab in this cohort of immunosuppressed and/or immunocompromised patients with advanced CSCC in real-world clinical settings are consistent with those observed in clinical trials that excluded these patients. ORR was 45.5% for this cohort, excluding patients who were enrolled after their third dose of cemiplimab.

### Example 3: C-POST Phase 3, Randomized, Double-Blind Study of Adjuvant Cemiplimab Versus Placebo Post Surgery and Radiation Therapy in Patients with High-Risk CSCC

This example relates to a planned study to evaluate cemiplimab as adjuvant treatment for patients with high-risk CSCC, based on surgical and clinicopathologic findings, who completed surgery and postoperative RT (minimum total dose 50Gy, within 10 weeks before randomization) (NCT03969004). This study is open for enrollment.

Patients with at least one of the following high-risk features are eligible: (1) nodal disease with (a) extracapsular extension and at least one node ≥20 mm or (b) at least three lymph nodes positive on surgical pathology report, regardless of extracapsular extension; (2) in-transit metastases; (3) T4 lesion; (4) perineural invasion; and (5) recurrent CSCC with at least one other risk factor. Patients with CSCC involvement in at least three lymph nodes (feature 1b) are included in the eligibility criteria. These criteria allow patients with chronic lymphocytic leukemia (CLL) who are not on active treatment to be enrolled.

The study is expected to enroll 412 patients from about 100 sites in North and South America, Europe, and Asia-Pacific regions. A primary objective of the study is to compare disease-free survival (DFS) of patients with high-risk CSCC treated with adjuvant cemiplimab, versus those treated with placebo, after surgery and radiation therapy (RT). Secondary objectives include: to compare the overall survival (OS) of high-risk CSCC patients treated with adjuvant cemiplimab, versus those treated with placebo, after surgery and RT; to compare the effect of adjuvant cemiplimab with that of placebo on patients' freedom from locoregional recurrence (FFLRR) after surgery and RT; to compare the effect of adjuvant cemiplimab with that of placebo on patients' freedom from distant recurrence (FFDR) after surgery and RT; to compare the effect of adjuvant cemiplimab with that of placebo on the cumulative incidence of second primary CSCC tumors (SPTs) after surgery and RT; to evaluate the safety of adjuvant cemiplimab and that of placebo in high-risk CSCC patients after surgery and RT; and to assess cemiplimab pharmacokinetics and immunogenicity in human serum.

Study Design: This study is a randomized, placebo-controlled, double-blind, multicenter, phase 3 study comparing cemiplimab, versus placebo, as adjuvant treatment for CSCC patients with features associated with high-risk of recurrent disease, who have completed surgery and post-operative RT. Ideally, post-operative RT should begin around 4 to 6 weeks following surgery if feasible. The study population comprises CSCC patients with high-risk features on surgical pathology who have completed surgery and post-operative RT.

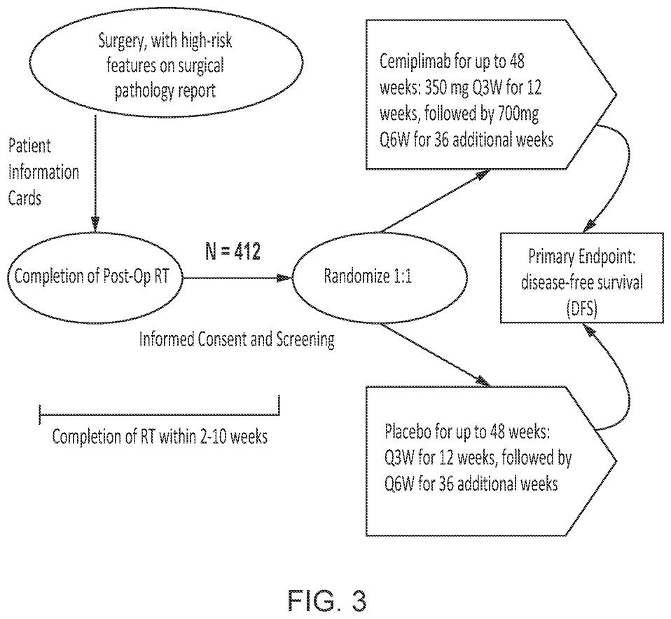

The study has two parts. In Part 1 (blinded), after a screening period of up to 28 days patients are randomly assigned 1:1 to receive cemiplimab 350 mg or placebo intravenously every 3 weeks for 12 weeks, followed by cemiplimab 700 mg or placebo every 6 weeks for 36 weeks for a total treatment period of up to 48 weeks. Patients will undergo post-treatment follow-up until disease recurrence or end of study. Part 1 of the study supports the primary endpoint. In optional Part 2 (unblinded), patients in the placebo arm who experience disease recurrence and patients in the cemiplimab arm who experience disease recurrence ≥3 months after completion of 48-week treatment in Part 1 are eligible to receive open-label cemiplimab 350 mg Q3W for up to 96 weeks. FIG. 3 shows an overview of the design for the blinded portion of the study (Part 1). Part 2 provides opportunity for additional cemiplimab treatment after recurrence but does not impact the primary endpoint of DFS.

Assigned Treatment in Part 1: Patients with high-risk features on surgical pathology who have completed post-operative RT and eligible after screening assessments will be randomized 1:1 to cemiplimab or placebo. The treatment schedule will be Q3W for 12 weeks followed by Q6W for 36 weeks for both treatment groups. Patients randomized to cemiplimab will be treated with cemiplimab 350 mg IV Q3W for 12 weeks followed by 700 mg Q6W for 36 weeks. Patients randomized to placebo will be treated at the same planned frequency, Q3W for 12 weeks followed by Q6W for 36 weeks. Both groups will be treated for a total duration of 48 weeks or until unacceptable toxicity, disease recurrence, death or withdrawal of consent. The first dose of cemiplimab or placebo will be administered within 5 days of randomization (not including day of randomization). Randomization will occur between 2 and 10 weeks after completion of RT. Patients will be evaluated in clinic prior to each cemiplimab or placebo treatment.

Post-Treatment Follow-up in Part 1: The follow-up period begins after discontinuation of treatment, either due to the completion of the planned 48-week treatment period or premature discontinuation of the treatment for any other reason (e.g., disease recurrence, or adverse event (AE) requiring discontinuation). During the first 2 years of follow-up, patients undergo clinical and radiological assessments every 4 months. During the third year of follow-up and thereafter, patients will undergo clinical and radiological assessments every 6 months.

Part 2 (Subsequent Cemiplimab Treatment): For patients who experience disease recurrence during Part 1 of the study, there is potential for optional subsequent cemiplimab therapy in Part 2 if requirements for disease recurrence are met. For patients assigned to placebo in Part 1, there is an option to “crossover” to cemiplimab in Part 2. For patients assigned to cemiplimab in Part 1, there is an option for cemiplimab “re-treatment” in Part 2. Cemiplimab treatment in Part 2 is only allowed for a patient's first recurrence on the study. Patients who enter Part 2 may receive cemiplimab 350 mg Q3W for up to 96 weeks or until disease progression, unacceptable toxicity, withdrawal of consent, death, or lost to follow-up. Patients on the cemiplimab arm who experience disease recurrence ≥3 months after completing cemiplimab treatment may be considered for subsequent cemiplimab treatment if the following conditions are met: documentation of disease recurrence ≥3 months (90 days±3 days) after completion of 48 weeks of planned cemiplimab treatment (even if 1 or more doses of planned cemiplimab treatment were missed during the 48-week treatment period); prior cemiplimab was not discontinued due to unacceptable toxicity; repeat screening procedures and continues to meet study eligibility criteria (with exception of select eligibility criteria).

Study Population: The target patient population will consist of adult high-risk CSCC patients who have undergone surgical resection followed by RT.

Inclusion Criteria: A patient must meet the following criteria to be eligible for inclusion in the study: (1) Men and women ≥18 years old (for Japan only, men and women ≥21 years old); (2) Patient with resection of pathologically confirmed CSCC (primary CSCC lesion only, or primary CSCC with nodal involvement, or CSCC nodal metastasis with known primary CSCC lesion previously treated within the draining lymph node echelon), with macroscopic gross resection of all disease; (3) High risk CSCC, as defined by at least 1 of the following: (a) Nodal disease with (i) extracapsular extension (ECE)* and at least 1 node ≥20 mm on the surgical pathology report, and/or (ii) ≥3 lymph nodes positive on surgical pathology report, regardless of ECE where ECE is defined as extension through the lymph node capsule into the surrounding connective tissue, with or without associated stromal reaction. Unambiguous evidence of gross ECE (defined as invasion of skin, infiltration of musculature/fixation to adjacent structures on clinical examination) is a sufficiently high threshold to classify these as ECE positive (AJCC, 2017); (b) In-transit metastases (ITM), defined as skin or subcutaneous metastases that are >2 cm from the primary lesion but are not beyond the regional nodal basin (Leitenberger, 2016); (c) T4 lesion, including HN lesions (AJCC, 2017) and non-HN lesions (UICC, Manual of Clinical Oncology, O'Sullivan B, et al. 9th ed., 2015); (d) Perineural invasion (PNI), defined as clinical and/or radiologic involvement of named nerves (UICC, 2015); (e) Recurrent CSCC, defined as CSCC that arises within the area of the previously resected tumor, plus at least 1 of the following additional features (AJCC, 2017): ≥N2b disease associated with the recurrent lesion; Nominal ≥T3 (recurrent lesion ≥4 cm in diameter or minor bone erosion or deep invasion >6 mm measured from the granular layer of normal adjacent epithelium); Poorly differentiated histology and ≥20 mm diameter of recurrent lesion. The recurrent tumor must be documented to be within the area of the previously resected CSCC by radial measurement of the greatest radius of the final defect, measured from the estimated center of the original surgical wound; (4) Completion of curative intent post-operative RT (concurrent chemoradiotherapy is acceptable) within 2 to 10 weeks of randomization. Patients must have received a minimum Biologically Equivalent Dose (BED) to the site of previous gross disease of 50 Gy (for head and neck primary sites and non-head and neck primary sites; (5) Eastern Cooperative Oncology Group performance status (ECOG PS) ≤1; (6) Adequate hepatic function: (a) Total bilirubin ≤1.5×upper limit of normal (ULN); (b) Transaminases (aspartate aminotransferase [AST] and alanine aminotransferase [ALT]) ≤3×ULN; (c) Alkaline phosphatase (ALP) ≤2.5×ULN; (7) Adequate renal function: Serum creatinine ≤1.5×ULN or estimated creatinine clearance (CrCl) >30 mL/min according to the method of Cockcroft and Gault; (8) Adequate bone marrow function: (a) Hemoglobin ≥9.0 g/dL; (b) Absolute neutrophil count (ANC) ≥1.0×10⁹/L; (c) Platelet count ≥75×10⁹/L; (9) Must be willing and able to provide informed consent signed by study patient or legally acceptable representative, as specified by health authorities and institutional guidelines; (10) Toxicities from radiotherapy must have resolved to grade 1 or less except the following toxicities, which must have resolved to grade 2 or less: dysgeusia, fatigue, xerostomia, trismus, alopecia, fibrosis, oropharyngeal mucositis, dermatitis, skin ulceration, or edema in radiated field; (11) Willing and able to comply with clinic visits and study-related procedures; (12) Able to understand and complete study-related questionnaires.

Exclusion Criteria: A patient who meets any of the following criteria will be excluded from the study: (1) Squamous cell carcinomas (SCCs) arising in non-cutaneous sites (e.g., dry red lip [vermillion], oral cavity, oropharynx, paranasal sinus, larynx, hypopharynx, nasopharynx, salivary gland, nasal mucosa, anogenital area, or SCC nodal metastasis with unknown primary). For patients parotid SCC, such patients are not considered “unknown primary” if the impression of the investigator is that the current parotid disease arose from a prior cutaneous lesion. Such patients may screen for the study; (2) Concurrent malignancy other than localized CSCC and/or history of malignancy other than localized CSCC within 3 years of date of randomization, except for tumors with negligible risk of metastasis or death, such as adequately treated (BCC) of the skin, carcinoma in situ of the cervix, or ductal carcinoma in situ of the breast, or low-risk early stage prostate adenocarcinoma (T1-T2ₐN0M0 and Gleason score ≤6 and prostate-specific antigen (PSA) ≤10 ng/mL) for which the management plan is active surveillance, or prostate adenocarcinoma with biochemical-only recurrence with documented PSA doubling time of >12 months for which the management plan is active surveillance (D'Amico, 2005; Pham, 2016); (3) Patients with hematologic malignancies (note: patients with chronic lymphocytic leukemia [CLL] are not excluded if they have not required systemic therapy for CLL within 6 months of enrollment); (4) Patients with history of distantly metastatic CSCC (visceral or distant nodal), unless the disease-free interval is at least 3 years (regional nodal involvement of disease in draining lymph node basin that was resected and radiated prior to enrollment will not be exclusionary, per exclusion criterion 2); (5) Ongoing or recent (within 5 years of randomization date) evidence of significant autoimmune disease that required treatment with systemic immunosuppressive treatments, which may suggest risk for immune-related adverse events (irAEs). The following are not exclusionary: vitiligo, childhood asthma that has resolved, type 1 diabetes, residual hypothyroidism that required only hormone replacement, or psoriasis that does not require systemic treatment; (6) Has participated in a study of an investigational agent or an investigational device within 4 weeks of the randomization date or five half-lives (whichever is longer), though patients who have received or are enrolled in a study involving treatment with an investigational immunoPET reagent are not excluded; (7) Receipt of a live vaccine within 28 days of the randomization date; (8) Has had prior systemic anti-cancer immunotherapy for CSCC. Examples of immune modulating agents include but are not limited to blockers of CTLA-4, 4-1BB (CD137), or OX-40, therapeutic vaccines, anti-PD-1/PD-L1 or PI3Kδ inhibitors; (9) Immunosuppressive corticosteroid doses (>10 mg prednisone daily or equivalent) within 4 weeks prior to the first dose of cemiplimab/placebo. Patients who require brief course of steroids (e.g., prophylaxis for imaging assessments due to hypersensitivity to contrast agents) are not excluded. People taking steroids for physiologic replacement (i.e., adrenal insufficiency) are NOT excluded; (10) Has received treatment with an approved anticancer systemic therapy within 4 weeks of the randomization date or has not yet recovered (i.e., ≤grade 1 or baseline) from any acute toxicities except for laboratory changes as described in inclusion criteria 6-8. Patients receiving bisphosphonates or denosumab are not excluded; (11) Prior allogeneic stem cell transplantation, or autologous stem cell transplantation; (12) Patients who have permanently discontinued anti-cancer immune modulating therapies due to drug-related toxicity; (13) Encephalitis, meningitis, or uncontrolled seizures in the year prior to screening/enrollment; (14) Patients with myocardial infarction within 6 months prior to the randomization date; (15) Any infection requiring hospitalization and/or intravenous antibiotic therapy within 2 weeks of the randomization date; (16) Active tuberculosis; (17) Uncontrolled infection with human immunodeficiency virus (HIV), hepatitis B or hepatitis C virus (HBV or HCV) infection; or diagnosis of immunodeficiency. Patients with known HIV infection who have controlled infection (undetectable viral load (HIV RNA PCR) and CD4 count above 350, either spontaneously or on a stable antiviral regimen) are permitted. For patients with controlled HIV infection, monitoring will be performed per local standards. Patients will be tested for HBV and HCV at screening. Patients with HBV (hepatitis B surface antigen positive; HepBsAg+) who have controlled infection (serum HBV DNA PCR that is below the limit of detection AND receiving anti-viral therapy for HBV) are permitted. Patients with controlled infections must undergo periodic monitoring of HBV DNA. Patients must remain on anti-viral therapy for at least 6 months beyond the last dose of investigational study drug. Patients who are HCV antibody positive (HCV Ab+) who have controlled infection (undetectable HCV RNA by PCR, either spontaneously or in response to a successful prior course of anti-HCV therapy) are permitted; (18) History of immune related pneumonitis within the last 5 years; (19) History of interstitial lung disease (e.g., idiopathic pulmonary fibrosis, organizing pneumonia) or active, noninfectious pneumonitis that required immune-suppressive doses of glucocorticoids to assist with management. A history of radiation pneumonitis in the radiation field is permitted as long as pneumonitis resolved ≥6 months prior to the randomization date; (20) History of documented allergic reactions or acute hypersensitivity reaction attributed to antibody treatments; (21) Known hypersensitivity or allergy to any of the excipients in the cemiplimab drug product; (22) Patients with a history of solid organ transplant (patients with prior corneal transplant(s) are not excluded); (23) Any medical co-morbidity, physical examination finding, or metabolic dysfunction, or clinical laboratory abnormality that, in the opinion of the investigator, renders the patient unsuitable for participation in a clinical trial due to high safety risks and/or potential to affect interpretation of results of the study; (24) Known psychiatric or substance abuse disorders that would interfere with participation with the requirements of the study; (25) Member of the clinical site study team or his/her immediate family; (26) Women with a positive serum β-human chorionic gonadotropin (HCG) pregnancy test at the screening/baseline visit. If positive, pregnancy must be ruled out by ultrasound for patient to be eligible; (27) Breastfeeding women; (28) Women of childbearing potential (WOCBP)* or sexually active men** whose partners are WOCBP, who are unwilling to practice highly effective contraception prior to the first dose of study therapy, during the study, and for at least 180 days after the last dose. *Highly effective contraceptive measures for women include: (a) Stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; (b) Intrauterine device (IUD); intrauterine hormone-releasing system (IUS); (c) Bilateral tubal ligation; (d) Vasectomized partner^{¥} and/or; (e) Sexual abstinence†‡. *Women of childbearing potential are defined as females who have had 1 episode of menses and have not yet reached menopause or have become surgically sterile, as below. A postmenopausal state is defined as no menses for 12 months without alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to Clinical Trial Facilitation Group (CTFG) guidance Pregnancy testing and contraception are not required for women with documented hysterectomy or tubal ligation. **Male study participants with WOCBP partners are required to use condoms unless they are vasectomized^{¥} or practice sexual abstinence†‡. Sexual abstinence is considered a highly effective method only if defined as refraining from heterosexual intercourse during the entire period of risk associated with the study treatments. Periodic abstinence (calendar, symptothermal, post-ovulation methods), withdrawal (coitus interruptus), spermicides only, and lactational amenorrhea method (LAM) are not acceptable methods of contraception. Female condom and male condom should not be used together. ^{¥}Vasectomized partner or vasectomized study participant must have received medical assessment of the surgical success.

Study Treatments: Cemiplimab will be supplied as a liquid in sterile, single-use vials. Each vial will contain cemiplimab at a concentration of 50 mg/mL. Placebo will be prepared using the same formulation as that used for cemiplimab without the addition of active substance. Placebo will be supplied as a liquid in sterile, single-use vials and administered in the same way as cemiplimab. Cemiplimab 350 mg or placebo will be administered in an outpatient setting as a 30-minute (±10 minutes) IV infusion every 3 weeks. Cycle length is 12 weeks (4 study treatments on a Q3W cycle). After the first cycle (after 12 weeks), the regimen will change to placebo Q6W or cemiplimab 700 mg IV as a 30-minute (±10 minutes) IV infusion (36 weeks at Q6W) for up to a total of 48 weeks. The planned treatment period in Part 1 of the study is 48 weeks.

Method of Treatment Assignment: Approximately 412 patients will be randomized in a 1:1 ratio in a blinded fashion to receive either cemiplimab or placebo according to a central randomization scheme. Randomization will be stratified by: Anatomic region of resected high-risk tumor: HN vs non-HN; Geographic Region: North America vs Australia/New Zealand vs Rest of World (ROW); High risk feature (nodal versus exclusively non-nodal). For example, if the patient meets high-risk criteria with both nodal and non-nodal features, they will be considered in the nodal stratum; ECOG PS: 0 vs 1; History of CLL: presence or absence. The stratification factors “high risk features”, “ECOG PS” and “history of CLL” are used for balancing treatment assignment only and will not be included in the statistical model for analysis of the primary endpoint.

Concomitant Medications and Procedures: Any treatment administered from the time of informed consent until 90 days after the last study treatment (cemiplimab or placebo) will be considered concomitant medication. This includes medications that were started before the study and are ongoing during the study, as well as any therapies started in the follow-up period to treat treatment related AEs.

Prohibited medications and procedures: While participating in this study (not including survival follow-up), a patient may not receive any of the following from the time of informed consent to the end of the follow-up period, unless otherwise specified below: Standard or investigational agent for treatment of a tumor other than cemiplimab or placebo, with the exception of those permitted below; Agents that block the PD-1/PD-L1 pathway (other than for patients who are assigned to receive cemiplimab in the study); Radiation therapy; Live vaccines for at least 3 months after the last dose of study drug.

Permitted Medications and Procedures: The following medications and procedures will be permitted, under the following conditions: Any medication required to treat an AE and/or irAE, including systemic corticosteroids; Systemic corticosteroids for physiologic replacement (even if >10 mg/day prednisone equivalents); A brief course of corticosteroids for prophylaxis or for treatment of non-autoimmune conditions; Oral contraceptives, hormone-replacement therapy, or other maintenance therapy may continue; Surgical resection of pre-malignant lesions or BCC lesions; Other medications and procedures may be permitted on an individual basis by the investigator and in consultation with the sponsor; Because this is an adjuvant study, surgery is not planned. However, if surgery for any emergent medical issue(s) is clinically indicated in the opinion of the investigator for an individual patient, this is allowed.

### Study Procedures

Procedures Performed at Screening/Baseline: The following procedures will be performed for the sole purpose of determining study eligibility or characterizing the baseline population: Serum β-HCG (test must be done ≤72 hours before the first dose); HBV, HCV, and HIV screening; Coagulation tests (International Normalized Ratio [INR] and activated partial thromboplastin time [aPTT]); Height measurement; Recording of medical history/oncology history and post-surgical RT information; Baseline radiological tumor assessment: Baseline imaging will be performed in concordance with on-study tumor assessments; Baseline circulating tumor DNA (ctDNA) detection; Post-Surgical Radiation Therapy.

Efficacy Procedures: Patients undergo imaging assessments at screening and at the end of each 12-week cycle during the planned treatment period of approximately 1 year (48 weeks). During each imaging assessment, the following radiologic imaging of the chest, abdomen, and pelvis is required. For patients in which the resected lesion was in the HN, imaging of the neck will be obtained. Options are: CT scan of chest/abdomen/pelvis (or CT chest and MRI abdomen/pelvis); For HN primaries: Neck CT and/or MRI; Other CT and/or MRI, as clinically indicated.

Recurrence is defined as the appearance of 1 or more new CSCC lesions (excluding SPT) that are locoregional or distant. Evidence of recurrence on imaging should be confirmed with a biopsy to obtain histologic or cytologic evidence of CSCC in all cases of suspected disease recurrence, unless biopsy is considered to pose an unacceptable safety risk.

Locoregional recurrence: Any of the following sites of disease recurrence: For HN CSCC, nodal or soft tissue recurrence above the clavicle; For non-HN CSCC, recurrence within the first draining nodal basin (or soft tissue associated within the first draining nodal basin) of the resected tumor; In-transit metastases, defined as skin or subcutaneous metastases that are >2 cm from the primary lesion but are not beyond the regional nodal basin.

Distant recurrence: Any of the following sites of disease recurrence: For HN CSCC, nodal recurrence below the clavicle; For non-HN CSCC, recurrence beyond the first draining nodal basin of the resected tumor bed. Recurrence in 2 nodal basins will be considered distant recurrence, even if contiguous (i.e., 2 mediastinal nodal basins, 2 pelvic nodal basins); Recurrence in non-nodal tissue (including, but not limited to, lung, liver, bone, brain); Epidermotropic metastases, defined as distant lesion(s) in the dermis without epidermal involvement.

Biopsies to document recurrent disease or new skin lesions: Biopsy to obtain histologic or cytologic evidence of CSCC should be attempted in all cases of suspected recurrence or suspected SPT unless biopsy is considered to pose an unacceptable safety risk in the opinion of the investigator (e.g., brain lesions). For SPTs, if biopsy is positive for CSCC, surgical removal of the lesion is recommended if possible (unless the biopsy was excisional).

Characterization of new skin lesions: In CSCC, the most common sites of metastases are lymph node and lung (Hillen et al., Eur J Cancer 2018; 96:34-43). New CSCC lesions in the skin usually are not metastatic lesions. With rare exceptions, new CSCC lesions in the skin are new primary tumors due to field cancerization from chronic UV-mediated skin damage (Christensen, F1000Res; 7:2018). In a previous randomized trial in advanced CSCC, recurrent skin disease and SPTs were recognized as distinct entities (Brewster et al., J Clin Oncol 2007; 25(15):1974-78). In this study, SPTs are non-metastatic CSCC lesions in the skin that can be managed by local modality therapy as part of routine clinical practice. However, there are two circumstances in which a new skin lesion in a CSCC patient could represent metastatic disease: (1) Epidermotropic metastases (EDM), defined as distant lesion(s) in the dermis without epidermal involvement (Skala et al., Histopathology 2018; 72(3):472-480; Weidner et al., Arch Dermatol 1985; 121(8):1041-1043). Although EDM are well descried in the melanoma literature, this would be a highly atypical pattern of recurrence in CSCC patients. However, EDM could occur in a CSCC patient because cutaneous metastases from squamous cancers arising in internal organs have been described (Bornkessel et al., Am J Dermatopathol 2006; 28(3):220-222.; Plataniotis et al., Br J Dermatol 1999; 141(3):579-580).

In-transit metastases are defined as cutaneous nodule(s) distinct from the primary tumor and occurring proximal to the first lymph node basin (Xu et al., Head Neck 2018; 40(7):1406-14). Of note, not all CSCC lesions arising in the skin proximal to the first lymph node basin are ITM. Due to chronic sun exposure, new lesions in sun-exposed skin may represent SPTs. The decision about whether to call such a lesion an SPT or an ITM will be based on the overall clinical presentation of the lesion. These ITMs are most often subcutaneous or dermal papules with occasional exophytic features (Carucci et al., Dermatol Surg 2004; 30(4 Pt 2):651-655).

In this study, a new CSCC lesion arising on the skin during the study period will be classified as an SPT unless the lesion represents ITM or EDM. Second primary CSCC tumors are not counted as events for the DFS endpoint. Patients who develop ITM or EDM will be considered to have experienced a DFS event. Second primary CSCC tumors arising during the study period should be treated with surgery or another permitted local modality. Permitted non-surgical local modalities for SPTs in this study are: topical 5-fluorouracil, topical imiquimod, and photodynamic therapy with topical aminolevulinic acid or methyl aminolevulinate (Christensen, 2018). For any cutaneous lesion that is resected during the course of the study, the following information will be recorded: histologic diagnosis, maximum diameter, and investigator's impression regarding whether lesion represents SPT or a disease recurrence (either ITM or EDM).

Patient-Reported Outcomes: Health related quality of life will be measured using the EORTC QLQ-C30 and EQ-5D-3L validated self-administered patient questionnaires. The EORTC QLQ-C30 covers the domains of global health status/quality of life, functional scales (physical, role, emotional, cognitive, and social), and symptom scales (fatigue, nausea and vomiting, pain, dyspnea, insomnia, appetite loss, constipation, diarrhea, and financial impact). The EQ-5D-3L is a validated measure that covers 5 items (mobility, self-care, usual activities, pain/discomfort, anxiety/depression) and 1 visual analogue scale. Patients will be asked to complete both questionnaires prior to any study procedures being performed at a given study visit.

Statistical Analysis: The results of the study are expected to show that cemiplimab prolongs DFS as compared with placebo. The full analysis set (FAS) includes all randomized patients. This is the intent-to-treat population. he FAS is based on the treatment allocated (as randomized). Efficacy endpoints will be analyzed using the FAS. The safety analysis set (SAF) includes all randomized patients who received any study drug; it is based on the treatment received (as treated). Treatment compliance/administration and all clinical safety variables will be analyzed using the SAF. The PK analysis population includes all patients who received any study drug and who had at least 1 non-missing result following the first dose of study drug. The anti-drug antibody (ADA) analysis set includes all patients who received any study drug and had at least 1 non-missing ADA result following the first study dose.

### Example 4: Study of Cemiplimab in Patients with Advanced Cutaneous Squamous Cell Carcinoma (CSCC) in a Real-World Setting

This example describes additional demographics, effectiveness, and safety results of cemiplimab in patients with advanced CSCC enrolled in the C.A.S.E. study (NCT03836105), described in Example 2.

Methods: C.A.S.E. is a prospective, real-world, multi-center study evaluating the effectiveness, safety, disease evolution, survivorship, and quality of life of advanced CSCC patients treated with cemiplimab. Patients received cemiplimab 350 mg intravenously every 3 weeks per routine standard of care. Demographics, disease characteristics, efficacy, and quality of life data were collected. Investigator assessment of objective response rate (ORR), survival, and safety was conducted. Data from a real-world general population of patients with advanced CSCC treated with cemiplimab are presented.

Results: 188 patients were enrolled in the C.A.S.E. study; the median age was 76.0 years (range: 33.0-98.0); 76.9% were male; and 90.9% were white; and 36 (19.1%) patients were considered immunocompromised (IC) or immunosuppressed (IS). Median duration of cemiplimab exposure for all patients was 22.1 weeks (quartile [Q]1-Q3: 9.1-46.4, range: 0-117). Efficacy was evaluated in patients enrolled prior to cycle 3 (n=164), where a clear treatment outcome could be established. ORR was 42.1% (95% confidence interval [CI]: 34.4%-50.0%). ORR for the IC/IS population (n=27) was 44.4% (95% CI: 25.5%-64.7%) and therefore efficacy appeared to be similar to that of the general population. Safety was evaluated in all patients included in the study; 8 (4.3%) patients experienced a treatment-related serious adverse event; and 47 (25.3%) patients experienced a treatment-related immune-related adverse event. In total, 95 (48.2%) patients discontinued treatment, with the most common reason being physician decision (22 [11.2%]). No treatment-related AEs that led to death were observed. Cemiplimab was well tolerated in IC/IS patients.

The safety, tolerability, and effectiveness of cemiplimab in this real-world study of patients with advanced CSCC are consistent with results observed in the registration clinical trial. (NCT02383212 and NCT02760498).

### REFERENCES

1. Alam et al., “Cutaneous squamous-cell carcinoma,” N Engl J Med, 2001; 344(13):975-83.
2. Athar et al., Arch Biochem Biophys. 2011; 508:159-163.
3. Burova et al. Mol Cancer Ther., 2017; 16:861-870.
4. Ikeda, “Phase II study of camptothecin in patients with squamous cell carcinoma of the skin,” Skin Cancer, 1993; 8:503-13.
5. Ikeda et al., “Peplomycin therapy for skin cancer in Japan,” Drugs Exp Clin Res 1986; 12(1-3):247-55.
6. Jennings et al., “The Sun Exposure and Behaviour Inventory (SEBI): validation of an instrument to assess sun exposure and sun protective practices,” J Eur Acad Dermatol Venereol, 2013; 27(6):706-15.
7. Karia et al., “Cutaneous squamous cell carcinoma: estimated incidence of disease, nodal metastasis, and deaths from disease in the United States, 2012,” J Am Acad Dermatol, 2013; 68(6):957-66.
8. LIBTAYO® (cemiplimab-rwlc) [prescribing information], Regeneron Pharmaceuticals, Inc., Tarrytown, NY (2021).
9. Lippman et al., “13-cis-retinoic acid and interferon alpha-2a: effective combination therapy for advanced squamous cell carcinoma of the skin,” J Natl Cancer Inst, 1992; 84(4):235-41.
10. Madan et al., “Non-melanoma skin cancer,” Lancet, 2010; 375(9715):673-85.
11. Maubec et al., “Phase II study of cetuximab as first-line single-drug therapy in patients with unresectable squamous cell carcinoma of the skin,” J Clin Oncol, 2011; 29(25):3419-26.
12. Migden et al., “PD-1 Blockade with Cemiplimab in Advanced Cutaneous Squamous-Cell Carcinoma,” N Engl J Med, 2018; 379(4):341-51.
13. Nakamura et al., “Platinum and anthracycline therapy for advanced cutaneous squamous cell carcinoma,” Int J Clin Oncol, 2013; 18(3):506-09.
14. Nottage et al., “A prospective study investigating the impact of definitive chemoradiation in locoregionally advanced squamous cell carcinoma of the skin,” Journal of Clinical Oncology, 2012; 30(15_suppl):8538.
15. Rhee et al., “Validation of a quality-of-life instrument for patients with nonmelanoma skin cancer,” Arch Facial Plast Surg, 2006; 8(5):314-18.
16. Rogers et al., “Incidence Estimate of Nonmelanoma Skin Cancer (Keratinocyte Carcinomas) in the U.S. Population, 2012,” JAMA Dermatol, 2015; 151(10):1081-86.
17. Samstein et al., “Locally advanced and unresectable cutaneous squamous cell carcinoma: outcomes of concurrent cetuximab and radiotherapy,” J Skin Cancer, 2014; 2014(284582).
18. Schmults et al., “Factors predictive of recurrence and death from cutaneous squamous cell carcinoma: a 10-year, single-institution cohort study,” JAMA Dermatol, 2013; 149(5):541-47.
19. Shin et al., “Phase II study of induction chemotherapy with paclitaxel, ifosfamide, and carboplatin (TIC) for patients with locally advanced squamous cell carcinoma of the head and neck,” Cancer, 2002; 95(2):322-30.
20. Williamson et al., “Pain: a review of three commonly used pain rating scales,” J Clin Nurs, 2005; 14(7):798-804.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method of treating or inhibiting the growth of a tumor, comprising:
(a) selecting a patient with cancer, wherein the patient is immunosuppressed or immunocompromised; and
(b) administering to the patient a therapeutically effective amount of a programmed death-1 (PD-1) inhibitor, wherein the PD-1 inhibitor is an antibody or antigen-binding fragment thereof that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2.

2. The method of claim 1, wherein the cancer is selected from anal cancer, bladder cancer, bone cancer, breast cancer, brain cancer, cervical cancer, colon cancer, colorectal cancer, endometrial cancer, esophageal cancer, head and neck cancer, kidney cancer, liver cancer, lung cancer, myeloma, ovarian cancer, pancreatic cancer, prostate cancer, salivary gland cancer, skin cancer, stomach cancer, testicular cancer, and uterine cancer.

3. The method of claim 1, wherein the cancer is skin cancer.

4. The method of claim 3, wherein the skin cancer is selected from cutaneous squamous cell carcinoma (CSCC), basal cell carcinoma (BCC), Merkel cell carcinoma, and melanoma.

5. The method of claim 4, wherein the skin cancer is CSCC.

6. The method of claim 5, wherein the skin cancer is metastatic or locally advanced CSCC and the patient is not a candidate for curative surgery or curative radiation.

7. The method of claim 4, wherein the skin cancer is BCC.

8. The method of claim 7, wherein the skin cancer is metastatic or locally advanced BCC, and wherein the patient has been previously treated with a hedgehog pathway inhibitor (HHI) or for whom HHI is not appropriate.

9. The method of claim 1, wherein the patient is immunocompromised or immunosuppressed due to a history of solid organ transplant.

10. The method of claim 1, wherein the patient is immunocompromised or immunosuppressed due to an autoimmune disease or disorder.

11. The method of claim 1, wherein the patient is immunocompromised or immunosuppressed due to a hematologic malignancy.

12. The method of claim 11, wherein the hematologic malignancy comprises a heme cancer.

13. The method of claim 12, wherein the heme cancer is chronic lymphocytic leukemia.

14. The method of claim 11, wherein the patient has undergone surgical resection followed by radiation therapy prior to administration of the PD-1 inhibitor.

15. The method of claim 1, wherein the cancer is CSCC and patient has at least one high-risk feature selected from: (1) nodal disease with (a) extracapsular extension and at least one node ≥20 mm or (b) at least three positive lymph nodes; (2) in-transit metastases; (3) T4 lesion; (4) perineural invasion; and (5) recurrent CSCC with at least one other risk factor.

16. (canceled)

17. (canceled)

18. The method of claim 1, wherein the anti-PD-1 antibody or antigen-binding fragment thereof comprises HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8.

19. The method of claim 18, wherein the anti-PD-1 antibody or antigen-binding fragment thereof comprises a HCVR comprising an amino acid sequence of SEQ ID NO: 1.

20. The method of claim 18, wherein the anti-PD-1 antibody or antigen-binding fragment thereof comprises a LCVR comprising an amino acid sequence of SEQ ID NO: 2.

21. The method of claim 18, wherein the anti-PD-1 antibody or antigen-binding fragment thereof comprises a HCVR/LCVR amino acid sequence pair of SEQ ID NOs: 1/2.

22. The method of claim 18, wherein the anti-PD-1 antibody comprises a heavy chain and a light chain, wherein the heavy chain has an amino acid sequence of SEQ ID NO: 9.

23. The method of claim 18, wherein the anti-PD-1 antibody comprises a heavy chain and a light chain, wherein the light chain has an amino acid sequence of SEQ ID NO: 10.

24. The method of claim 18, wherein the anti-PD-1 antibody comprises a heavy chain and a light chain, wherein the heavy chain has an amino acid sequence of SEQ ID NO: 9 and the light chain has an amino acid sequence of SEQ ID NO: 10.

25. The method of claim 1, wherein the PD-1 inhibitor is cemiplimab.

26. The method of claim 18, wherein the PD-1 inhibitor is an anti-PD-1 antibody or antigen-binding fragment thereof comprising a HCVR with 90% sequence identity to SEQ ID NO: 1.

27. The method of claim 18, wherein the PD-1 inhibitor is an anti-PD-1 antibody or antigen-binding fragment thereof comprising a LCVR with 90% sequence identity to SEQ ID NO: 2.

28. The method of claim 18, wherein the PD-1 inhibitor is an anti-PD-1 antibody or antigen-binding fragment thereof comprising a HCVR with 90% sequence identity to SEQ ID NO: 1, and a LCVR with 90% sequence identity to SEQ ID NO: 2.

29. (canceled)

30. (canceled)

31. The method of claim 1, wherein the administration of the PD-1 inhibitor promotes tumor regression, reduces tumor cell load, reduces tumor burden, and/or prevents tumor recurrence in the patient.

32. The method of claim 1, wherein the administration of the PD-1 inhibitor leads to at least one effect selected from an increase in one or more of overall response rate, progression-free survival, overall survival, complete response, partial response, and stable disease.

33. The method of claim 1, wherein the administration of the PD-1 inhibitor does not cause an adverse event related to the immunosuppressed or immunocompromised condition of the patient.

34. The method of claim 1, wherein the PD-1 inhibitor is administered as a monotherapy.

35. The method of claim 1, wherein the PD-1 inhibitor is administered in combination with an additional therapeutic agent or therapy selected from surgery, radiation, an anti-viral therapy, photodynamic therapy, HHI therapy, imiquimod, a programmed death ligand-1 (PD-L1) inhibitor, a lymphocyte activation gene 3 (LAG3) inhibitor, a cytotoxic T-lymphocyte-associated protein 4 (CTLA-4) inhibitor, a glucocorticoid-induced tumor necrosis factor receptor (GITR) agonist, a T-cell immunoglobulin and mucin domain containing protein-3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an antagonist of another T-cell co-inhibitor or ligand, a CD20 inhibitor, an indoleamine-2,3-dioxygenase (IDO) inhibitor, a CD28 activator, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an agonist to a co-stimulatory receptor, an antibody to a tumor-specific antigen, a vaccine, an adjuvant to increase antigen presentation, an oncolytic virus, a cytotoxin, a chemotherapeutic agent, platinum-based chemotherapy, a tyrosine kinase inhibitor, an IL-6R inhibitor, an IL-4R inhibitor, an IL-10 inhibitor, a cytokine, an antibody drug conjugate (ADC), chimeric antigen receptor T cells, an anti-inflammatory drug, a non-steroidal anti-inflammatory drug (NSAID), and a dietary supplement.

36. The method of claim 1, wherein the PD-1 inhibitor is administered as one or more doses, wherein each dose is administered every two weeks, three weeks, four weeks, five weeks or six weeks.

37. The method of claim 1, wherein the PD-1 inhibitor is administered as two or more doses, wherein each dose is administered every three weeks.

38. The method of claim 1, wherein the PD-1 inhibitor is administered at a dose of 5 mg to 800 mg.

39. The method of claim 1, wherein the PD-1 inhibitor is administered at a dose of 200 mg, 250 mg, 350 mg, or 700 mg.

40. The method of claim 1, wherein the PD-1 inhibitor is administered at a dose of 1 mg/kg to 20 mg/kg of the patient's body weight.

41. The method of claim 1, wherein the PD-1 inhibitor is administered at a dose of 1 mg/kg, 3 mg/kg or 10 mg/kg of the patient's body weight.

42. The method of claim 1, wherein the PD-1 inhibitor is administered intravenously or subcutaneously.

43. (canceled)

44. A kit comprising a programmed death 1 (PD-1) inhibitor in combination with written instructions for use of a therapeutically effective amount of the PD-1 inhibitor for treating or inhibiting the growth of a tumor in an immunosuppressed or immunocompromised cancer patient, wherein the PD-1 inhibitor is an antibody or antigen-binding fragment thereof that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2.
